(19) United States
(12) Patent Application Publication
(10) Pub. No.: US 2026/0248645 A1
(43) Pub. Date: Aug. 27, 2026

(54) IMPROVED HEAT EXCHANGE CATHETERS FOR USE WITH A PATIENT BODY TEMPERATURE MANAGEMENT SYSTEM

(71) Applicant: ZOLL Circulation, Inc., San Jose, CA (US)

(72) Inventors: Adrienne E. Prasad, Santa Clara, CA (US); Masoumeh Mafi, Mountain View, CA (US); Jeremy T. Dabrowiak, Santa Clara, CA (US); George L. Walls, San Jose, CA (US); John T. Buckley, San Jose, CA (US); Mark G. Mitchell, San Jose, CA (US); Richard A. Helkowski, Redwood City, CA (US)

(73) Assignee: ZOLL Circulation, Inc., San Jose, CA (US)

(21) Appl. No.: 19/116,077

(22) PCT Filed: Sep. 28, 2023
(86) PCT No.: PCT/US23/75335

§ 371 Date: Mar. 27, 2025

Related U.S. Application Data
(60) Provisional application No. 63/377,727, filed on Sep. 30, 2022.

(51) Int. Cl. A61F 7/12; A61F 7/00; A61M 25/10

(52) U.S. Cl. CPC A61F 7/123; A61M 25/1002; A61F 2007/0054; A61F 2007/0092; A61F 2007/126; A61M 2025/1004; A61M 25/1011; A61M 2205/366; A61M 2210/12

(57) ABSTRACT

In an illustrative embodiment, a heat exchange catheter includes a catheter body and a heat exchange region. The catheter body includes a delivery lumen, a return lumen, an inner wall separating the delivery and return lumens, and a side wall defining at least one opening in communication with the delivery lumen. The inner wall defines an opening in communication with the delivery and return lumens. A first end of the heat exchange region is coupled to the delivery lumen of the catheter body through the at least one opening. The heat exchange region is configured to be in fluid communication with the delivery and return lumens of the catheter body to receive a working fluid. The opening of the inner wall is located such that the working fluid flowing through the delivery lumen of the catheter body is bifurcated between the heat exchange region and the return lumen.

### CROSS-REFERENCE TO RELATED APPLICATION(S)

This application claims the benefit of and priority to U.S. Provisional Patent Application No. 63/377,727, filed on Sep. 30, 2022, the entire disclosure of which is incorporated herein by reference.

### TECHNICAL FIELD

The present disclosure relates generally to systems and heat exchange catheters for controlling a patient's body temperature.

### BACKGROUND

In various clinical situations, it is desirable to warm, cool or otherwise control the body temperature of a subject. For example, hypothermia can be induced in humans and some animals for the purpose of protecting various organs and tissues (e.g., heart, brain, kidneys) against the effects of ischemic, anoxic or toxic insult. For example, animal studies and/or clinical trials suggest that mild hypothermia can have neuroprotective and/or cardioprotective effects in animals or humans who suffer from ischemic cardiac events (e.g., myocardial infract, acute coronary syndromes, etc.), postanoxic coma after cardiopulmonary resuscitation, traumatic brain injury, stroke, subarachnoid hemorrhage, fever, and neurological injury.

One method for inducing hypothermia is by intravascular or endovascular temperature management wherein a heat exchange catheter is inserted into a blood vessel and a heat exchange fluid is circulated through a heat exchange region positioned on the portion of the catheter that is inserted in the blood vessel. As the heat exchange fluid circulates through the catheter's heat exchange region, it exchanges heat with blood flowing past the heat exchange region in the blood vessel. Such technique can be used to cool the subject's flowing blood thereby resulting in a lowering of the subject's core body temperature to some desired target temperature. Endovascular temperature management is also capable of warming the body and/or of controlling body temperature to maintain a monitored body temperature at some selected temperature. If a controlled rate of re-warming or re-cooling from the selected target temperature is desired, that too can be accomplished by carefully controlling the amount of heat added or removed from the body and thereby controlling the temperature change of the patient.

### SUMMARY

The present disclosure describes temperature management systems and heat exchange catheters that may have a smaller or reduced profile relative to conventional heat exchange catheters and/or improved heat exchange efficiency and/or improved cooling and/or warming power output. The temperature management system is configured to control a temperature of a patient's body using a heat exchange device such as a catheter or a surface pad. The temperature management system may be configured to monitor how a heat exchange device is operating and/or to control the temperature of the patient's body (also called treatment or temperature management treatment or heat exchange treatment). The temperature management system includes a console that is coupled to the heat exchange device. The console includes an interface for receiving or coupling to a fluid loop configured to supply the heat exchange device with heating or cooling working fluid (e.g., saline). The interface may provide instructions, indicators, hardware elements, and/or a graphical interface to assist a user in configuring and priming the fluid loop for operation. In certain implementations, the fluid loop may include a plurality of flexible tubes configured to connect a coil, a spike, an air trap cylinder, a pump tube, and/or a heat exchange device into the fluid loop.

The implementations described herein can provide one or more advantages. The present disclosure describes various heat exchange catheters that demonstrate a practical approach to meeting the performance requirements and overcoming usability challenges associated with managing a patient's body temperature. A heat exchange catheter having a reduced or smaller profile relative to conventional heat exchange catheters allows placement thereof in various vessels and through various insertion sites. For example, such heat exchange catheter may be used as a peripherally inserted central catheter (PICC), which, in turn, enables non-physician clinicians to place the heat exchange catheter into a vasculature of a patient, e.g., into the vasculature in a patient's arm. A heat exchange catheter having improved heat exchange efficiency and/or cooling or warming power output, whether it be a catheter with a reduced profile compared to traditionally sized heat exchange catheters, or a traditional or larger catheter, may be more practical and versatile, allowing for controlled cooling or warming, and for use in treating patients who have experienced various conditions such as, e.g., a stroke or heart attack.

In certain implementations, a heat exchange catheter having a smaller or reduced profile relative to conventional heat exchange catheters may suffer from buildup of backpressure, which, in turn, may reduce a flow rate of a working fluid circulating through the heat exchange catheter. Reduction in the flow rate of the working fluid may reduce heat exchange efficiency and/or power output of the heat exchange catheter. However, a heat exchange catheter may be provided with bifurcated fluid paths to reduce buildup of backpressure. This improves the flow rate of the working fluid through the heat exchange catheter, which, in turn may improve heat exchange efficiency and/or increase heat exchange power output of the heat exchange catheter. In addition, a tube or a tubular balloon may be supported about a catheter body of the heat exchange catheter in a helical manner which may enable reduction in a diameter of the heat exchange catheter to allow for insertion in smaller vessels while maintaining or enhancing heat exchange power output. Such a configuration may also facilitate or improve manufacturability thereof.

In certain implementations, a heat exchange catheter may further include an expandable inner wall that separates a supply lumen and a return lumen within a catheter body of the heat exchange catheter. The expandable inner wall may dynamically enlarge the supply lumen or the return lumen of the catheter body without enlarging the overall diameter of the catheter body of the heat exchange catheter. In this manner, a reduced profile of the heat exchange catheter may be achieved.

In certain implementations, a heat exchange catheter may include a free floating balloon coupled to a catheter body. Under such a configuration, a need for a support structure or elongated portion of the catheter body to support the balloon may be eliminated. The free floating balloon may have a reduced diameter when deflated and/or may be held within the catheter shaft or against an outer wall of the catheter body in its deflated state, which allows for a reduced profile of the heat exchange catheter, allowing for insertion of the catheter into smaller vessels or through smaller insertion sites.

Improving heat exchange efficiency and/or power output of a heat exchange catheter need not be limited to the heat exchange catheters having a smaller or reduced profile relative to conventional heat exchange catheters. By improving heat exchange efficiency and/or power output of even the heat exchange catheters having traditional or larger sizes that are placed by a physician at insertion sites including femoral or subclavian arteries and internal or external jugular veins, target temperatures may be reached more quickly and/or greater control of a patient's temperature may be exerted. For example, the heat exchange efficiency and/or power output may be increased by reducing thermal shadowing. This may be accomplished by angularly offsetting and/or axially displacing projections of a heat exchange region of a heat exchange catheter.

The implementations described herein can include one or more of the following aspects or embodiments. In general, one or more features of an embodiment associated with one aspect of the disclosure may be combined with another aspect of the disclosure.

In accordance with an aspect of the present disclosure, a heat exchange catheter is insertable into a vasculature of a patient. The heat exchange catheter includes a catheter body and a heat exchange region. The catheter body includes a delivery lumen, a return lumen, an inner wall separating the delivery and return lumens, and a side wall defining at least one opening in communication with the delivery lumen. The inner wall defines an opening in communication with the delivery and return lumens. The delivery lumen and the return lumen are configured to receive a working fluid therethrough. The heat exchange region includes a first end and a second end. The first end of the heat exchange region is coupled to the delivery lumen of the catheter body through the at least one opening. The heat exchange region is configured to be in fluid communication with the delivery and return lumens of the catheter body to receive the working fluid and thereby effect heat exchange with blood flowing through the vasculature of the patient. The opening of the inner wall is located such that the working fluid flowing through the delivery lumen of the catheter body is bifurcated between the heat exchange region and the return lumen. The inner wall may be provided within the catheter body. The inner wall may extend in a longitudinal direction along the catheter body.

In some embodiments, the opening in the inner wall may be upstream of the heat exchange region.

In some embodiments, the heat exchange region may project through the at least one opening such that the heat exchange region is supported about the catheter body.

In some embodiments, the working fluid flowing through the heat exchange region may bypass a portion of the delivery lumen of the catheter body.

In some embodiments, the catheter body may further include a pair of plugs. The pair of plugs may be disposed in the delivery lumen to establish a fluid-tight seal against the delivery lumen such that the working fluid is directed through the heat exchange region.

In some embodiments, the catheter body may further define a working lumen. The working lumen may be dimensioned to receive a guidewire therethrough.

In some embodiments, the catheter body may have a diameter ranging between about 5 Fr (1.67 mm) and about 6 Fr (2 mm).

In some embodiments, the heat exchange region may be an inflatable balloon.

In accordance with an aspect of the present disclosure, a heat exchange catheter is insertable into a vasculature of a patient. The heat exchange catheter includes a catheter body and a heat exchange region. The catheter body includes a delivery lumen, a return lumen, and an inner wall therebetween. The inner wall is radially expandable into the delivery lumen or the return lumen whereby the inner wall expands into the return lumen to enlarge the delivery lumen when a working fluid is supplied through the catheter body, and the inner wall expands into the delivery lumen to enlarge the return lumen when the working fluid is drained through the catheter body. The heat exchange region is coupled to the catheter body and configured to be in fluid communication with the delivery and return lumens of the catheter body to effect heat exchange with blood of the patient.

In some embodiments, the heat exchange region and the catheter body may be configured to be peripherally inserted.

In some embodiments, a diameter of the heat exchange region may be less than about 6 Fr (2 mm).

In some embodiments, the catheter body may further define a working lumen.

In some embodiments, the catheter body may further define a working lumen through the inner wall such that the working lumen is movable with the inner wall as a single construct.

In some embodiments, the catheter body may further define a working lumen extending through the catheter body. The working lumen may be stationary relative to the inner wall.

In some embodiments, the catheter body may be monolithically formed as a single construct.

In some embodiments, the heat exchange region may be or may comprise an inflatable balloon.

In some embodiments, the heat exchange region may be configured to inhibit reverse flow of the heat exchange fluid into the delivery lumen.

In some embodiments, the heat exchange region may be supported about the catheter body in a deflated state.

In accordance with an aspect of the present disclosure, a heat exchange catheter is insertable into a vasculature of a patient. The heat exchange catheter includes a catheter body and an inflatable heat exchange balloon. The catheter body includes a delivery lumen, a return lumen, and a distal end. The delivery and return lumens are configured to receive a working fluid therethrough. The inflatable heat exchange balloon is in communication with the delivery and return lumens of the catheter body and configured to receive the working fluid and thereby effect heat exchange with blood flowing through the vasculature of the patient. The inflatable heat exchange balloon is coupled to the distal end of the catheter body such that the heat exchange balloon is transitionable between a deflated state in which the heat exchange balloon is proximal to the distal end of the catheter body and an inflated state in which the inflatable heat exchange balloon is deployed distal to the distal end of the catheter body such that the heat exchange balloon floats within the vasculature of the patient.

In some embodiments, the heat exchange catheter may further include a sleeve. The sleeve may be configured to cover the inflatable heat exchange balloon during insertion of the heat exchange catheter into the vasculature of the patient.

In some embodiments, the inflatable heat exchange balloon may include a distal end portion having a first diameter in the inflated state. The catheter body may have a second diameter that is larger or smaller than the first diameter.

In some embodiments, the catheter body may include a channel configured to receive the inflatable heat exchange balloon in the deflated state. The inflatable heat exchange balloon in the deflated state may be bonded to the catheter body and may be configured to be displaced distally by a stylet.

In accordance with an aspect of the present disclosure, a heat exchange catheter is insertable into a vasculature of a patient. The heat exchange catheter includes a catheter body and a heat exchange region. The catheter body includes a delivery lumen and a return lumen. The delivery and return lumens are configured to receive a working fluid. The heat exchange region includes an elongate body in communication with the delivery and return lumens of the catheter body and configured to receive the working fluid and thereby effect heat exchange with blood flowing through the vasculature of the patient. The elongate body of the heat exchange region includes a plurality of first heat exchange projections protruding radially outwards from the catheter body. At least one first heat exchange projection of the plurality of first heat exchange projections includes a surface transverse to a direction of a flow of the blood and the at least one first heat exchange projection is angularly offset from another first heat exchange projection of the plurality of first heat exchange projections in a single flow path.

In some embodiments, the working fluid in the single flow path may flow in a general direction of the flow of the blood. The single flow path may be provided by or within the plurality of first heat exchange projections. The single flow path may provide at least a portion of a supply fluid path or at least a portion of a return fluid path.

In some embodiments, adjacent first heat exchange projections of the plurality of first heat exchange projections of the heat exchange region may define an acute angle. The angularly offset may be defined in a plane that is orthogonal to a longitudinal axis of the catheter body.

The plurality of first heat exchange projections may extend in a direction of the longitudinal axis of the catheter body. In some embodiments, the adjacent first heat exchange projections of the heat exchange region may be axially displaced along the direction of the flow of the blood.

In some embodiments, the heat exchange region may further include a plurality of second heat exchange projections.

In some embodiments, the working fluid may flow through the plurality of second heat exchange projections in a general direction opposite of the direction of the flow of the blood.

In some embodiments, the heat exchange region may be an inflatable balloon. At least one second heat exchange projection of the plurality of second heat exchange projections may include a surface transverse to a direction of a flow of the blood. The at least one second heat exchange projection may be angularly offset from another second heat exchange projection of the plurality of second heat exchange projections in a single flow path.

In some embodiments, the inflatable balloon may be secured to the catheter body such that supply of the working fluid causes the inflatable balloon to deploy radially outwards.

In some embodiments, the heat exchange catheter may further include a tubular mesh coupled to the catheter body. The heat exchange region may be supported on the tubular mesh.

In some embodiments, adjacent first heat exchange projections in the single flow path may be angularly offset from each other.

In some embodiments, each first heat exchange projection may be a loop.

In accordance with an aspect of the present disclosure, a heat exchange catheter is insertable into a vasculature of a patient. The heat exchange catheter includes a catheter body and a heat exchange region. The catheter body includes a delivery lumen and a return lumen. The heat exchange region includes a body portion defining a longitudinal axis. The body portion is in communication with the delivery and return lumens of the catheter body to receive a working fluid and thereby effect heat exchange with blood flowing through the vasculature of the patient. The body portion of the heat exchange region includes a plurality of first heat exchange portions (or projections) such that the working fluid flows through the plurality of first heat exchange portions in a single flow path. At least two first heat exchange portions of the plurality of first heat exchange portions define respective planes transverse to a direction of a flow of the blood. The at least two first heat exchange portions are axially displaced and angularly offset from each other to reduce thermal shadowing.

In some embodiments, the working fluid may flow through the single flow path in a direction opposite of the direction of the flow of the blood.

In some embodiments, the working fluid may flow through the single flow path in the direction of the flow of the blood.

In some embodiments, the heat exchange region may further include a plurality of second heat exchange portions such that the working fluid flows through the plurality of second heat exchange portions in a direction opposite of the direction of the flow of the blood.

In some embodiments, the plurality of second heat exchange portions may be configured to be downstream of the plurality of first heat exchange portions.

In some embodiments, each first heat exchange portion of the plurality of first heat exchange portions may extend radially outwards from the longitudinal axis of the heat exchange region.

In some embodiments, the plurality of first heat exchange portion in the single flow path may define a hypotrochoid profile.

In some embodiments, parts of the at least two first heat exchange portions may be in a superposed relation.

In some embodiments, the heat exchange region may be an inflatable balloon.

In some embodiments, each first heat exchange portion of the plurality of first heat exchange portions may define a plane substantially orthogonal to the direction of the flow of the blood.

In some embodiments, each first heat exchange portion of the plurality of first heat exchange portions may have an annular profile.

In accordance with an aspect of the present disclosure, a heat exchange catheter is insertable into a vasculature of a patient. The heat exchange catheter comprises a catheter body and an inflatable heat exchange region. The catheter body includes a delivery lumen, a return lumen, and a distal end. The delivery and return lumens are configured to receive a working fluid therethrough. The inflatable heat exchange region is coupled to the distal end of the catheter body. The inflatable heat exchange region includes a plurality of ridges (or projections or protrusions) arranged about the catheter body. Each ridge of the plurality of ridges is in fluid communication with other ridges of the plurality of ridges. The inflatable heat exchange region is in communication with the delivery and return lumens of the catheter body and configured to receive the working fluid and thereby effect heat exchange with blood flowing through the vasculature of the patient. The heat exchange region is transitionable between a deflated state in which the plurality of ridges is configured to be wrapped around the catheter body and an inflated state in which the plurality of ridges extends radially outwardly with respect to the catheter body.

In some embodiments, the plurality of ridges may be configured to receive the working fluid in series.

In accordance with an aspect of the present disclosure, a heat exchange catheter is insertable into a vasculature of a patient. The heat exchange catheter comprises a catheter body and an inflatable heat exchange region. The catheter body includes a delivery lumen, a return lumen, and a distal end. The delivery and return lumens are configured to receive a working fluid therethrough. The inflatable heat exchange region is coupled to the distal end of the catheter body. The inflatable heat exchange region includes an inflatable chamber and a plurality of ridges that extends radially outwardly from the inflatable chamber. The inflatable heat exchange region is in communication with the delivery and return lumens of the catheter body and configured to receive the working fluid and thereby effect heat exchange with blood flowing through the vasculature of the patient.

In some embodiments, the inflatable chamber and the plurality of ridges may be formed as a single construct.

In some embodiments, the inflatable chamber and the plurality of ridges may be configured to be in fluid communication.

In some embodiments, the plurality of ridges may be uniformly arranged about the inflatable chamber.

In accordance with an aspect of the present disclosure, a heat exchange catheter is insertable into a vasculature of a patient. The heat exchange catheter includes a catheter body and a heat exchange region. The catheter body includes a delivery lumen, a return lumen, an inner wall separating the delivery and return lumens, and a side wall. The side wall defining at least one opening in communication with the delivery lumen.

The inner wall may define an opening between, or in communication with, the delivery and return lumens. The opening of the inner wall may be located such that the working fluid flowing through the delivery lumen of the catheter body is bifurcated between the heat exchange region and the return lumen.

The delivery lumen may be defined between the inner wall and a first portion of the side wall. The return lumen may be defined between the inner wall and a second portion of the side wall that is different to the first portion of the side wall. At least a portion of the inner wall may be configured to be deformable or flexible such that movement of the inner wall changes the relative volumes of the delivery lumen and the return lumen. The inner wall may be configured to radially expand into the delivery lumen or the return lumen whereby, for example, the inner wall may expand into the return lumen to enlarge the delivery lumen when a working fluid is supplied through the catheter body, and the inner wall may expand into the delivery lumen to enlarge the return lumen when the working fluid is drained through the catheter body.

In accordance with an aspect of the present disclosure, a heat exchange catheter is insertable into a vasculature of a patient. The heat exchange catheter includes a catheter body and an inflatable heat exchange region. The catheter body includes a delivery lumen, a return lumen. The inflatable heat exchange region is in communication with the delivery and return lumens of the catheter body.

The inflatable heat exchange region may be coupled to the distal end of the catheter body.

The inflatable heat exchange region may include a plurality of ridges arranged about the catheter body. The heat exchange region may be transitionable between a deflated state in which the plurality of ridges may be configured to be wrapped around the catheter body and an inflated state in which the plurality of ridges may be configured to extends radially outwardly with respect to the catheter body. The ridged may be configured to expand radially outwards when the heat exchange region transitions from the deflated state to the inflated state.

The inflatable heat exchange region may include an inflatable chamber and a plurality of ridges that extends radially outwardly from the inflatable chamber.

In accordance with an aspect of the present disclosure, a heat exchange catheter is insertable into a vasculature of a patient. The heat exchange catheter includes a catheter body and a heat exchange region. The catheter body includes a delivery lumen and a return lumen. The heat exchange region may include a plurality of first heat exchange projections protruding outwardly from the catheter body. At least one first heat exchange projection of the plurality of first heat exchange projections includes a surface transverse to a direction of a flow of the blood (or transverse to a longitudinally direction of the catheter) and the at least one first heat exchange projection is angularly offset from another first heat exchange projection of the plurality of first heat exchange projections in a single flow path.

The heat exchange region may have a longitudinal axis. The heat exchange region may be in communication with the delivery and return lumens of the catheter body to receive a working fluid and thereby effect heat exchange with blood flowing through the vasculature of the patient. The heat exchange region may include a plurality of first heat exchange portions arranged such that the working fluid flows through the plurality of first heat exchange portions in a single flow path. At least two first heat exchange portions of the plurality of first heat exchange portions may define respective planes transverse to a direction of a flow of the blood. The at least two first heat exchange portions may be axially displaced and angularly offset from each other to reduce thermal shadowing.

According to an aspect of the disclosure, there is provided a heat exchange catheter which includes a catheter body and a heat exchange region. The catheter body includes a delivery lumen, a return lumen, an inner wall separating the delivery and return lumens, and a side wall defining at least one opening in communication with the delivery lumen. The inner wall defines an opening in communication with the delivery and return lumens. A first end of the heat exchange region is coupled to the delivery lumen of the catheter body through the at least one opening. The heat exchange region is configured to be in fluid communication with the delivery and return lumens of the catheter body to receive a working fluid. The opening of the inner wall is located such that the working fluid flowing through the delivery lumen of the catheter body is bifurcated between the heat exchange region and the return lumen.

In general, a reference to a first feature being in communication with a second feature may mean that the first feature is in fluid communication with the second feature.

The details of one or more embodiments are set forth in the accompanying drawings and the description below. Other features and advantages will be apparent from the description and drawings, and from the claims.

### BRIEF DESCRIPTION OF THE DRAWINGS

The accompanying drawings, which are incorporated in and constitute a part of the specification, illustrate one or more embodiments and, together with the description, explain these embodiments. The above and other aspects and features of this disclosure will become more apparent in view of the following detailed description when taken in conjunction with the accompanying drawings wherein like reference numerals identify similar or identical elements. The heat exchange catheters disclosed herein are described in detail with reference to the drawings, in which like reference numerals designate identical or corresponding elements in each of the several views. The terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features.

FIG. 1 is a schematic diagram of a temperature management system illustrating use thereof.

FIG. 2 is a schematic diagram of a heat exchange catheter for use with the temperature management system of FIG. 1 in accordance with an embodiment of the present disclosure, illustrating use of the heat exchange catheter in a patient.

FIG. 3 is a cross-sectional view of the heat exchange catheter of FIG. 2.

FIG. 4 is a perspective view of a heat exchange catheter for use with the temperature management system of FIG. 1.

FIG. 4A is a partial side cross-sectional view of the heat exchange catheter of FIG. 4.

FIG. 5A is a perspective view of a heat exchange catheter for use with the temperature management system of FIG. 1 in accordance with another embodiment of the present disclosure.

FIG. 5B is a perspective view of the heat exchange catheter of FIG. 5A, illustrating a heat exchange region collapsed against an outer surface of a catheter body in the absence of a fluid.

FIG. 6 is a perspective view of a heat exchange catheter for use with the temperature management system of FIG. 1 in accordance with another embodiment of the present disclosure.

FIG. 7 is a cross-sectional view of the heat exchange catheter of FIG. 6, illustrating a catheter body of the heat exchange catheter in a neutral position.

FIG. 8 is a cross-sectional view of the heat exchange catheter of FIG. 6, illustrating the catheter body with an enlarged supply lumen.

FIG. 9 is a cross-sectional view of the heat exchange catheter of FIG. 6, illustrating the catheter body of the heat exchange catheter with an enlarged return lumen.

FIG. 10 is a cross-sectional view of a heat exchange catheter for use with the temperature management system of FIG. 1 in accordance with another embodiment of the present disclosure.

FIG. 11 is a cross-sectional view of the heat exchange catheter of FIG. 10, illustrating a catheter body with an enlarged supply lumen.

FIG. 12 is a cross-sectional view of the heat exchange catheter of FIG. 10, illustrating the catheter body with an enlarged return lumen.

FIG. 13 is a cross-sectional view of a heat exchange catheter for use with the temperature management system of FIG. 1 in accordance with another embodiment of the present disclosure, illustrating a catheter body with an enlarged supply lumen.

FIG. 14 is a cross-sectional view of the heat exchange catheter of FIG. 13, illustrating the catheter body with an enlarged return lumen.

FIG. 15A is a perspective view of a heat exchange catheter for use with the temperature management system of FIG. 1 in accordance with another embodiment of the present disclosure.

FIG. 15B is a partial perspective view of the heat exchange catheter of FIG. 15A, illustrating use of the heat exchange catheter with a sheath.

FIG. 15C is a partial perspective view of the heat exchange catheter of FIG. 15A, illustrating a heat exchange region in a fully inflated and floating state.

FIG. 16 is a partial perspective view of the heat exchange catheter for use with the temperature management system of FIG. 1 in accordance with yet another embodiment of the present disclosure.

FIG. 17 is a partial perspective view of the heat exchange catheter of FIG. 16, illustrating use of the heat exchange catheter with the sheath.

FIG. 18 is a partial perspective view of the heat exchange catheter of FIG. 16, illustrating the heat exchange region in a fully inflated and floating state.

FIG. 19 is a partial side view of a heat exchange catheter for use with the temperature management system of FIG. 1 in accordance with another embodiment of the present disclosure.

FIG. 20 is another partial side view of the heat exchange catheter of FIG. 19.

FIG. 21 is a perspective view of a heat exchange region for use with the temperature management system of FIG. 1 in accordance with another embodiment of the present disclosure.

FIG. 22 is a partial perspective view of a heat exchange catheter for use with the temperature management system of FIG. 1 in accordance with another embodiment of the present disclosure.

FIG. 23 is a partial side view of a heat exchange catheter for use with the temperature management system of FIG. 1 in accordance with another embodiment of the present disclosure.

FIG. 24 is a partial side cross-sectional view of a heat exchange region of the heat exchange catheter of FIG. 23, illustrating fluid conduits.

FIG. 24A is a partial side cross-sectional view of a heat exchange region of the heat exchange catheter of FIG. 23, illustrating fluid conduits in accordance with another embodiment of the disclosure.

FIG. 25 is a side view of the heat exchange region of FIG. 23, illustrating the heat exchange region in a deflated state.

FIG. 26 is a cross-sectional view of the heat exchange region of FIG. 23.

FIG. 27 is a perspective view of a heat exchange catheter for use with the temperature management system of FIG. 1 in accordance with yet another embodiment of the present disclosure.

FIG. 28 is a perspective view of a heat exchange catheter in accordance with yet another embodiment of the present disclosure.

FIG. 29 is a cross-sectional view of the heat exchange catheter of FIG. 28 cut along section line 30-30 of FIG. 28.

FIG. 30 is a cross-sectional view of the heat exchange catheter of FIG. 28 in a deflated state.

FIG. 31 is a cross-sectional view of the heat exchange catheter of FIG. 28 cut along section line 31-31 of FIG. 28.

FIG. 32 is a perspective view of a heat exchange region of a heat exchange catheter in accordance with yet another embodiment of the present disclosure.

FIG. 33 is a partial side cross-sectional view of the heat exchange region of FIG. 32.

FIG. 34 is a cross-sectional view cut along section line 34-34 of FIG. 33.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

When a patient in critical condition, e.g., a patient who has experienced cardiac arrest, needs temperature management treatment to control their body temperature, it is necessary to administer such treatment as quickly and efficiently as possible to optimize the effectiveness of the treatment and the patient's chances for recovery. The improved temperature management systems, catheters, and heat exchange fluid loops described herein include several features that facilitate ease of insertion and removal of a heat exchange catheter to and from a patient and effective heat exchange between the heat exchange catheter and the patient allowing the caregiver to provide the patient with treatment as quickly and efficiently as possible.

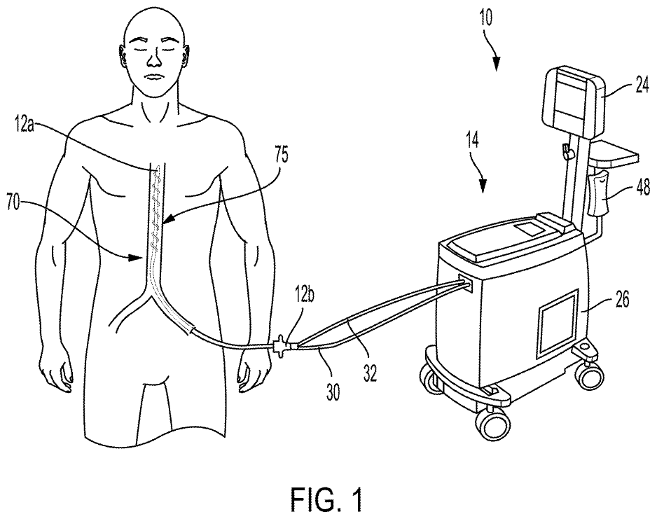

FIG. 1 shows one implementation of a temperature management system 10. The temperature management system 10 is configured to control a temperature of a patient's body using a heat exchange device 70, e.g., an intravascular heat exchange catheter that is inserted into a vasculature of a patient, or a heat exchange pad (not shown) positionable against a surface of the patient. The heat exchange device 70 is connected to an extracorporeal control console 14 by way of an inflow line 30 and an outflow line 32 such that a pump (not shown) within the extracorporeal control console 14 may circulate temperature-controlled heat exchange fluid through a heat exchange region 75 of the heat exchange device 70.

The extracorporeal control console 14 includes hardware for managing the patient temperature. The hardware generally includes components that make up an interface of the extracorporeal control console 14, which are configured to interface with components of a fluid loop that is configurable for connecting to the heat exchange device 70. The fluid loop includes the tubing assembly which facilitates connection of the heat exchange device 70 with the extracorporeal control console 14. The extracorporeal control console 14 may include a main housing 26 and a console head 24. The main housing 26 may contain various apparatuses and circuitry for warming/cooling heat exchange fluid, e.g., coolant, refrigerant, saline, to controlled temperature(s) and for pumping such warmed or cooled heat exchange fluid through the heat exchange device 70 to effectively modify and/or control the subject's body temperature. The console head 24 includes a display device or user interface, such as a touch screen system, whereby certain information may be input by, and certain information may be displayed to, users of the temperature management system 10. The user interface enables a user to input data or control signals to the temperature management system 10 and to present information, such as treatment data, indicative of treatment of the patient using the temperature management system 10. The user interface 24 may present or display various types of information associated with temperature management treatment of the patient and use of the temperature management system, including, e.g., patient data, operational data, priming instructions, and/or setup instructions.

One or more temperature sensors 12a, 12b may be located on the heat exchange device 70 and/or may be located on a separate device positioned elsewhere in the body, e.g., in the esophagus or rectum of the patient. The temperature sensor(s) 12a, 12b may be positioned on or in the subject's body to measure the temperature of all or part of the body where it is desired to effect temperature modification or control. On the main housing 26 there are provided connection ports for connection of temperature sensor(s) 12a, 12b that may be inserted through a heat exchange device 70 as well as other connection ports for connection of additional or alternative types of temperature sensors and/or other apparatus. In an embodiment, the temperature sensor 12a, 12b may be connected by way of a temperature lead (not shown), or alternatively by wireless connectivity, to the controller. The controller within the extracorporeal console 14 receives signals from the temperature sensor(s) 12a, 12b indicating the currently sensed body temperature. A desired target temperature may be input via the user interface and the controller will then issue control signals to the heater or cooler and/or pump to adjust the temperature and/or the flowrate of the heat exchange fluid in an effort to attain and/or maintain the target body temperature. The user interface displays system information and also receives user input as well as sensor data. A source of the heat exchange fluid 48, such as a bag or container of sterile 0.9% NaCl solution, is connected by tubing to the heating or cooling device. In an embodiment, the heat exchange device 70 and/or the temperature sensors 12a, 12b may be disposable items intended for a single use. The control console 14 may be a non-disposable system intended for multiple uses and to interface with the single-use device(s).

Exemplary temperature management systems and extracorporeal control consoles include the Thermogard XP® and Thermogard HQ™ manufactured by ZOLL Circulation. Reference may be made to U.S. patent application Ser. No. 17/561,512, the entire disclosure of which is incorporated herein by reference, for a detailed discussion of an example of an extracorporeal control console 14 and the tubing assembly. Further reference may be made to U.S. Pat. No. 11,185,440, the entire disclosure of which is incorporated herein by reference.

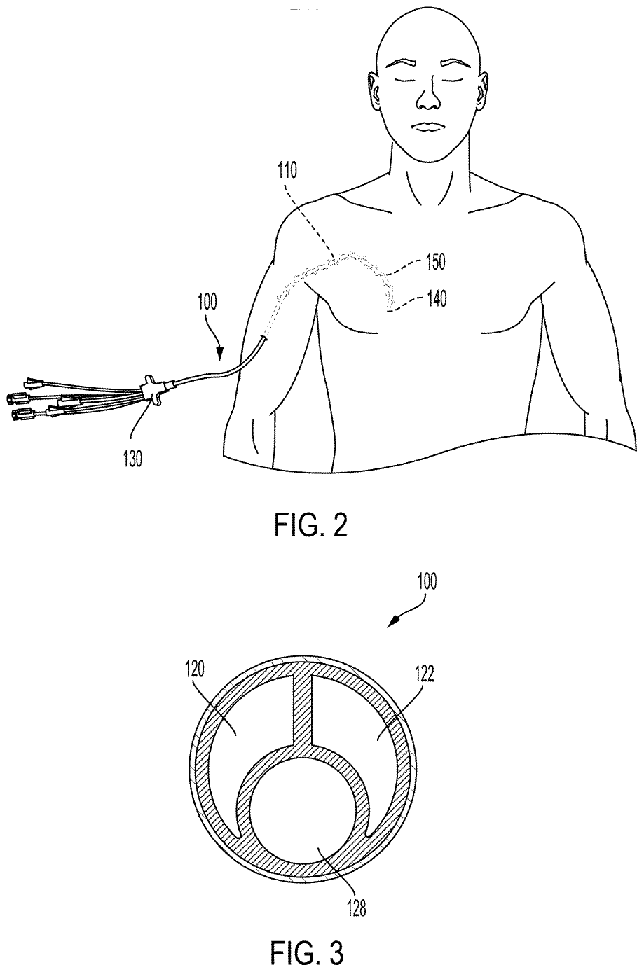

As shown in FIG. 1, generally, the heat exchange device 70 in the form of a heat exchange catheter may be inserted into the patient via various insertion sites including femoral or subclavian arteries or internal or external jugular veins by a physician. The blood, e.g., cooled by the heat exchange region 75 of the heat exchange device 70, may be processed by the heart and provided to the body in oxygenated form to be used as a conductive medium to cool the body. Inserting the heat exchange catheter through these insertion sites requires placement of the heat exchange catheter by a physician, thereby limiting the number of caregivers who can insert such heat exchange catheters. To this end, FIG. 2 shows an example of a heat exchange catheter 100 for use with the temperature management system 10 (FIG. 1) in accordance with the present disclosure. The heat exchange catheter 100 may provide a smaller or reduced profile compared to traditional heat exchange catheters, which are typically inserted in the vessels mentioned above. The catheter 100 may also provide enhanced heat exchanged capacity by improving flow of the heat exchange fluid circulating therethrough. In addition, the reduced profile of the heat exchange catheter 100 enables placement by a non-physician clinician, in other insertion sites, e.g., in vessels located in the arm. In this manner, the heat exchange catheter 100 may be used as a peripherally inserted central catheter. The heat exchange catheter 100 is placed in the vasculature of the patient to exchange heat with the blood. Such heat exchange causes a cooling or warming of the blood flowing through the vasculature. Such a method and device may therapeutically be used to induce and recover from an artificial state of hypothermia, or cool or warm patients suffering from a stroke, heart attack or other conditions.

With reference to FIGS. 2 and 3, there is provided the exemplary heat exchange catheter 100 in accordance with the present disclosure. The heat exchange catheter 100 is insertable into a vasculature of a patient in order to intravascularly lower or raise the temperature of a body of a patient. In particular, the heat exchange catheter 100 has a reduced profile relative to conventional heat exchange catheters to enable a non-physician clinician to place the heat exchange catheter 100. The heat exchange catheter 100 may be placed in a peripheral vessel. The heat exchange catheter 100 may include a catheter body 110, a heat exchange region 150 coupled to the catheter body 110, a manifold 130 disposed at a proximal portion of the catheter body 110, and a distal tip 140, e.g., an atraumatic distal tip or cap, located at a distal portion of the catheter body 110. The heat exchange catheter 100 having the reduced profile may have an outer diameter of less than 12 F. In an embodiment, an outer diameter ranges between about 5 F and about 6 F. The catheter body 110 may include a delivery lumen 120, a return lumen 122, and a working channel 128. The delivery lumen 120 and the return lumen 122 are configured to receive a heat exchange fluid therethrough. The working channel 128 is configured to receive, e.g., a guidewire, or alternatively serve as an infusion channel to provide medicament to the patient. The heat exchange region 150 may be formed of noncompliant polymeric material, such as polyethylene terephthalate (PET), Pebax®, Polyolefin, Polyurethane and/or Nylon or other suitable compliant or noncompliant material. The heat exchange region 150 may be constructed from a high conductivity material such that the temperature of an exterior surface thereof may reach very close to the temperature of the heat exchange fluid flowing through the heat exchange region 150. The heat exchange region in this and the other embodiments described herein may take on various configurations. For example, the heat exchange region may include one or more elongated tubing and/or balloons. In certain examples, the tubing or balloon may form one or more loops, helixes, cylinders or other configurations.

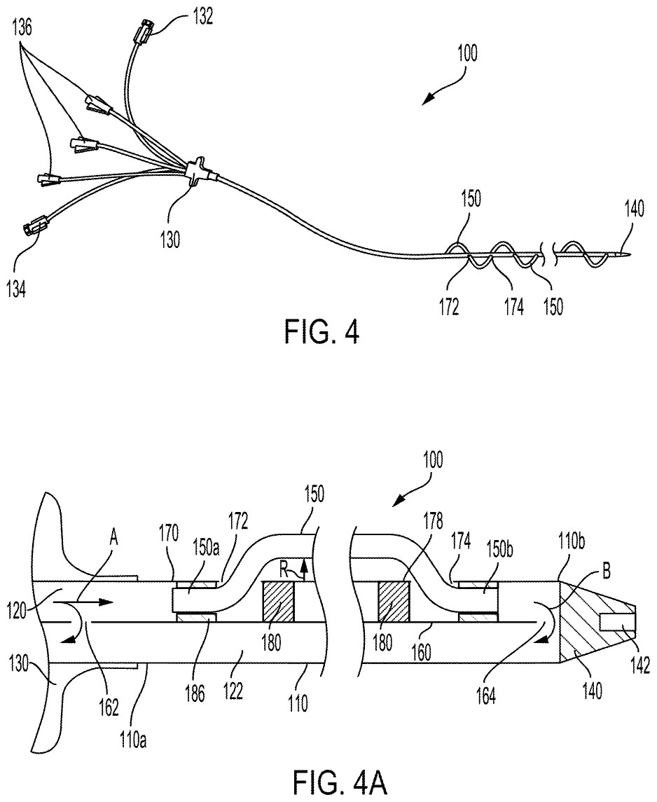

With reference to FIGS. 4 and 4A, the manifold 130 may include an inflow connector 132, an outflow connector 134, and one or more infusion lines 136 coupled to one or more working lumens or channels. At least one working lumen may serve as a guidewire lumen to facilitate insertion and positioning of the catheter and/or may be used after insertion of the catheter for delivery of fluids, medicaments, or other devices. Optionally, the catheter may be inserted using an introducer. For example, as shown in FIG. 1, in some embodiments, the temperature sensor 12a may be inserted through the working lumen of the catheter and advanced out of the distal end opening to a location beyond the distal end of the catheter body 110. In an embodiment, a portion of the manifold 130 may be overmolded onto the catheter body 110. The inflow connector 132 of the manifold 130 is in communication with the delivery lumen 120 of the catheter body 110 and is connectable to the extracorporeal control console 14 via an outflow line 32 (FIG. 1) to supply the heat exchange fluid through the heat exchange region 150 of the heat exchange catheter 100. The outflow connector 134 of the manifold 130 is in communication with the return lumen 122 of the catheter body 110 and is connectable to the extracorporeal control console 14 via an inflow line 30 (FIG. 1) to return the heat exchange fluid to the extracorporeal control console 14. The one or more infusion lines 136 are in communication with one or more working lumens or working channels. While FIG. 3 shows a single working fluid channel or lumen 128, the catheter body 110 may include a plurality of working fluid channels or lumens. The one or more working fluid channels or lumens may be utilized to supply, e.g., medicament or a guidewire to the patient.

With particular reference to FIG. 4A, the distal tip 140 is disposed at the distal portion 110b of the catheter body 110. The distal tip 140 defines an opening 142 in communication with the working channel 128 (FIG. 3) of the catheter body 110. The working channel 128 of the catheter body 110 terminates at the opening 142 in the distal end 110b of the catheter body 110. In an embodiment, the distal tip 140 may have a tapered or atraumatic configuration to facilitate insertion and inhibit trauma to the patient. Under such a configuration, a guidewire (not shown) may be inserted through the working lumen 128 of the catheter body 110 and extend out of the opening 142 of the distal tip 140 to facilitate insertion and position of the heat exchange catheter 100 within the vasculature. After the insertion of the heat exchange catheter 100, the working channel 128 may be used for infusion of fluids, medicaments, or other devices. In some embodiments, a temperature sensor may be inserted through the working channel 128 of the catheter body 110 and advanced out of the opening 142 of the distal tip 140 to a location beyond the distal end 110b of the catheter body 110.

In an example, as shown in FIG. 4A, the catheter body 110 may include an inner wall 160 and an outer wall 170. The outer wall 170 defines at least a first opening 172 and a second opening 174. The first and second openings 172, 174 are in direct communication with the delivery lumen 120 of the catheter body 110. Under such a configuration, a heat exchange region 150 may be secured to the catheter body 110 through the first and second openings 172, 174. In an embodiment, the heat exchange region 150 may be a tube or an inflatable balloon configured to receive the heat exchange fluid therethrough. The heat exchange region 150 has a proximal portion 150a and a distal portion 150b. The proximal portion 150a may extend through or otherwise be coupled to the first opening 172 and is secured to the catheter body 110 in a sealing relation. Similarly, the distal portion 150b is received through or otherwise be coupled to the second opening 174 and is also secured to the catheter body 110 in a sealing relation. In an embodiment, the proximal and distal portions 150a, 150b of the heat exchange region 150 may each be provided with a seal 186 to form a fluid tight seal against the catheter body 110 to inhibit infusion of the heat exchange fluid into the patient and to maintain a closed loop heat exchange fluid circuit. Under such a configuration, the heat exchange fluid flows through the delivery lumen 120 of the catheter body 110 and is directed through the heat exchange region 150. In addition, the delivery lumen 120 may be further provided with at least one plug 180 that forms a fluid tight seal against the catheter body 110. The at least one plug 180 may be disposed between the first and second openings 172, 174 to inhibit flow of blood into the delivery lumen 120 of the catheter body 110. Under such a configuration, the heat exchange fluid flows through the delivery lumen 120 and is directed through the heat exchange region 150. In certain embodiments, the heat exchange region 150 may be secured to the catheter body 110 through first and second openings 172, 174, wherein both the first and second slots 172, 174 are in an outer wall of the delivery lumen 120. In this configuration, heat exchange fluid flows through the delivery lumen 120 of the catheter body 110 and is directed through the heat exchange region 150 and from the heat exchange region 150 back into the delivery lumen 120. The heat exchange fluid flows from the delivery lumen to a second heat exchange region or to the return lumen. In certain embodiments, the heat exchange region 150 may be secured to the catheter body 110 through first and second openings 172, 174, wherein the first opening 172 is in an outer wall of the delivery lumen 120 and the second opening 174 is in an outer wall of the return lumen 122. In this configuration, heat exchange fluid flows through the delivery lumen 120 of the catheter body 110 and is directed through the heat exchange region 150 and flows from the heat exchange region 150 directly into the return lumen 120.

With continued reference to FIG. 4A, the inner wall 160 separates the delivery lumen 120 and the return lumen 122. The inner wall 160 may be formed as an insulating divider to thermally separate the delivery lumen 120 and the return lumen 122. Due to the decreased need for insulation, the diameter of the catheter body 110 may be made smaller. The enhanced heat transfer characteristics of the interior and/or exterior surface of the heat exchange region 150 also allow the heat exchange fluid to be delivered to the heat exchange region 150 at, e.g., lower pressure. The inner wall 160 defines a first opening 162 and a second opening 164. The first opening 162 is upstream of the proximal portion 150a of the heat exchange region 150 or the first opening 172. The first opening 162 provides direct communication between the delivery lumen 120 and the return lumen 122. The heat exchange fluid flowing through the delivery lumen 120 of the catheter body 110 is bifurcated between the heat exchange region 150 and the return lumen 122 of the catheter body 110 such that a portion of the heat exchange fluid supplied to the delivery lumen 120 is directed to bypass the heat exchange region 150 and return back to the extracorporeal control console 14 (FIG. 1) via the inflow line 30 (FIG. 1). Such a configuration reduces buildup of backpressure and enhances flow rate of the heat exchange fluid, which, in turn, may enhance heat exchange capability of the heat exchange region 150 despite the heat exchange catheter 100, e.g., the heat exchange region 150, having a reduced profile. The second opening 164 of the inner wall 160 may be disposed downstream of the second opening 174 of the catheter body 110 or the heat exchange region 150 such that the heat exchange fluid flowing out of the heat exchange region 150 flows from the delivery lumen 120 to the return lumen 122. In an embodiment, the second opening 164 may be disposed in the distal portion 110b of the catheter body 110. In certain embodiments, for example, where the second opening 174 is in an outer wall of the return lumen 122, the catheter body 110 may not include a second opening 164. For example, heat exchange fluid may flow through the heat exchange region 150 from the delivery lumen 120 of the catheter body 110, and from the heat exchange region 150 the heat exchange fluid flows directly into the return lumen 120.

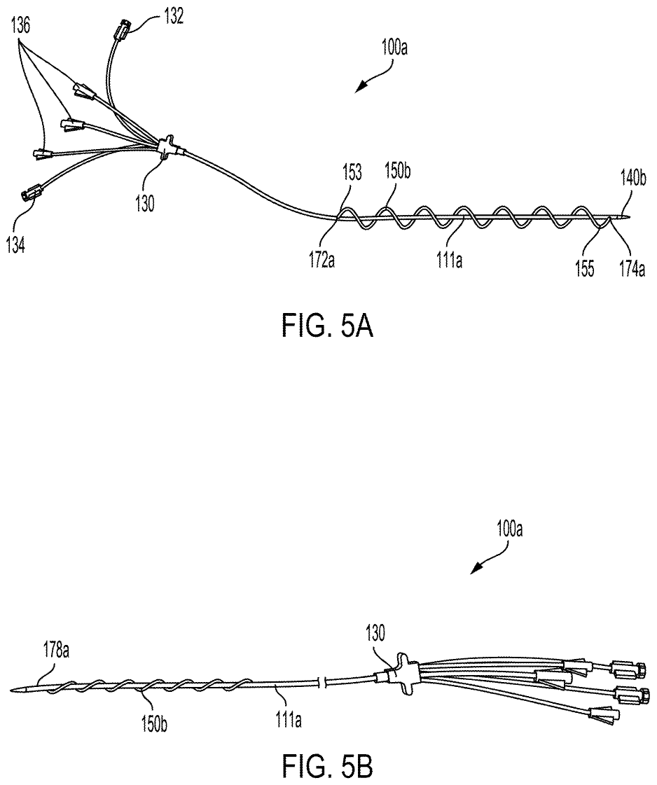

In certain embodiments, additional openings may be defined in the inner wall 160 (FIG. 4A). For example, the additional openings may be defined to provide additional relief from backpressure buildup and further enhance flow rate of the heat exchange fluid. Additional openings may be utilized when a single or plurality of heat exchange regions are provided. Each heat exchange region 150 may be a tube or an inflatable balloon configured to receive the heat exchange fluid therethrough. In an embodiment, the heat exchange region 150 may be formed of an elastic or flexible material such that in the absence of the heat exchange fluid, the heat exchange region 150, or a portion 155 of the heat exchange region 150, may be supported against an outer surface 178 (FIG. 5) of the outer wall 170 interposed between the first and second openings 172, 174 (FIG. 5).

In the example shown in FIG. 4, the heat exchange region 150 is shown to be received through a plurality of first and second openings 172, 174. However, it is further contemplated that in certain embodiments, the proximal and distal ends 153, 155 of a single heat exchange region 150b of a heat exchange catheter 100a may be coupled to the catheter body 111a through respective first and second openings 172a, 174b, as shown in FIG. 5A. Under such a configuration, the heat exchange region 150b may wrap around the catheter body between the first and second openings 172a, 174b. While a single heat exchange region 150 is shown to extend through a plurality of first and second openings 172, 174 in FIG. 4, it is further contemplated that in certain embodiments a plurality of heat exchange regions 150 may be utilized such that each heat exchange region 150 extends through the corresponding first and second openings 172, 174. As described hereinabove with respect to the heat exchange catheter 100, the heat exchange catheter 100a may also have a reduced profile relative to conventional heat exchange catheters to enable a non-physician clinician to place the heat exchange catheter 100. The heat exchange region 150b may be collapsed against an outer surface of a catheter body 111a in the absence of a heat exchange fluid to further reduce the profile of the heat exchange catheter 100a to facilitate, e.g., insertion or removal, of the heat exchange catheter 100a into or from the vasculature of the patient.

With reference back to FIG. 4A, when the heat exchange fluid is supplied to the heat exchange region 150, the heat exchange region 150, or the portion 155 of the heat exchange region 150, inflates and extends radially outwards in a direction of an arrow “R”. Such a configuration further reduces the profile of the heat exchange catheter 100 during insertion and removal of the heat exchange catheter 100 into and from the patient compared to the profile of the heat exchange catheter 100 when the heat exchange region 150 is in an inflated or extended state. In an embodiment, the heat exchange region 150 or the portion 155 of the heat exchange region 150 may initially lay against or be collapsed against the outer surface 178 and expand radially outwards when the heat exchange fluid is supplied to the heat exchange region 150. The heat exchange region 150 is configured to be in fluid communication with the delivery and return lumens 120, 122 of the catheter body 110 to receive the heat exchange fluid and thereby effect heat exchange with blood flowing through the vasculature of the patient.

In an embodiment, graduated distance markings may optionally be formed on the proximal portion 110a of the catheter body 110 to indicate a length of the heat exchange catheter 100 that is indwelling in the body of the patient at any particular time. Also, an optional proximal radiographic marker and an optional distal radiographic marker may be placed on the catheter body 110 to facilitate radiographic determination of the location of the heat exchanging region 150 within a body of a patient. The heat exchange region 150 may dwell within the blood vessel for extended periods of time, such as 24-48 hours or even longer up to 7 days. Thus, it may be desirable to treat the surfaces of the heat exchange region 150 to avoid clot formation.

In use, the heat exchange catheter 100 is connected to the extracorporeal control console 14. The reduced profile of the heat exchange catheter 100 relative to conventional heat exchange catheters may allow the heat exchange catheter 100 to be introduced into the patient's body without an introducer sheath. Eliminating the use of an introducer sheath may reduce the time needed for catheter placement and occurrence of complications associated therewith. For example, it may allow for a smaller insertion site into the patient's vessel, which may reduce risk of blood loss or infection The heat exchange region 150 is placed in the blood vessel and the heat exchange fluid such as saline or other aqueous solution may be circulated through the heat exchange region 150 of the heat exchange catheter 100 in closed loop. The heat exchange fluid flows through the delivery lumen 120 of the catheter body 110 and is bifurcated between the heat exchange region 150 and the return lumen 122 of the catheter body 110 such that a portion of the heat exchange fluid supplied to the delivery lumen 120 is directed to bypass the heat exchange region 150 and return back to the extracorporeal control console 14 (FIG. 1) via the inflow line 30 (FIG. 1). By providing two pathways for fluid to flow (one through the heat exchange region and one through the gap 162 into the return lumen and back through the inflow line 30) back pressure in the heat exchange catheter 100 is reduced and a higher flow rate of the heat exchange fluid can be maintained. In the embodiment shown in FIG. 4A, at the distal portion 150b of the heat exchange region 150, the heat exchange fluid exits the delivery lumen 120 of the catheter body 110 and also enters the return lumen 122 of the catheter body 110 in this portion of the heat exchange catheter 100. As the heat exchange fluid flows through the heat exchange region 150, heat is transferred between the heat exchange fluid and blood flowing against the surface of the heat exchange region 150. The heat exchange fluid flows through the return lumen 122 of the catheter body 110 and back to the extracorporeal control console 14 via the inflow line 30. The cooling can be adjusted by increasing or decreasing the flow rate of the saline. The temperature along the heat exchange region 150 may be monitored to reach or maintain the desired cooling effect. The heat exchange catheter 100 may be left in place to provide cooling as needed. Thereafter, the heat exchange catheter 100 is removed from the patient upon completion of the treatment. In certain embodiments, the heat exchange region 150 may be configured to provide heat exchange fluid flow in a proximal to a distal direction, and the return lumen 122 is configured to return the heat exchange fluid from the distal to the proximal direction. In other embodiments, the delivery lumen 122 may be configured to provide heat exchange fluid flow in the proximal to the distal direction, and the heat exchange region 150 may be configured to return the heat exchange fluid from the distal to the proximal direction. In other embodiments, the heat exchange region 150 may include a first portion configured to provide heat exchange fluid flow in the proximal to the distal direction and a second portion configured to return the heat exchange fluid from the distal to the proximal direction, whereby the heat exchange region 150 enables a bi-directional fluid flow through the heat exchange region 150. In an embodiment, the heat exchange region 150b may initially lay against or be collapsed against the outer surface 178a as shown in FIG. 5B and expand radially outwards when the heat exchange fluid is supplied to the heat exchange region 150b as shown in FIG. 5B.

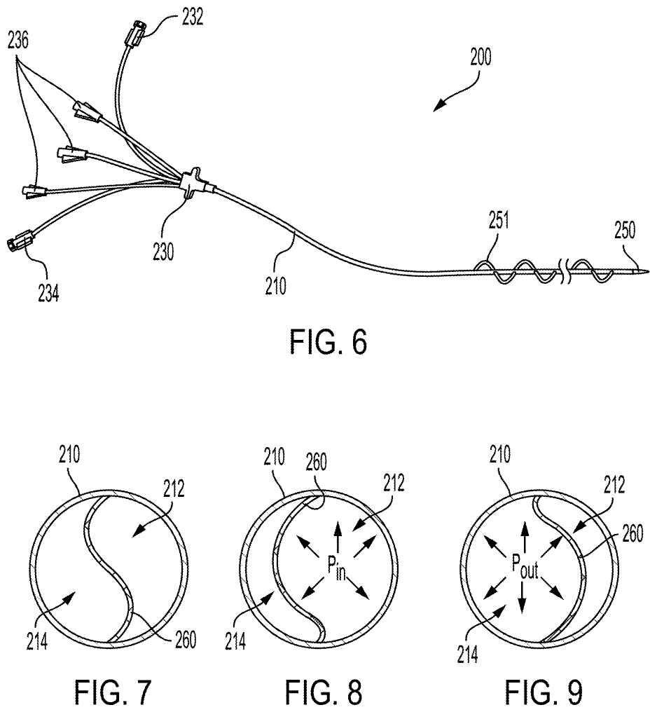

With reference now to FIG. 6, there is illustrated a heat exchange catheter 200 in accordance with another embodiment of the present disclosure. Portions of the heat exchange catheter 200 such as, e.g., a manifold 230 and a heat exchange region 250, may be similar to the manifold 130 (FIG. 2) and the heat exchange region 150 (FIG. 2) of the heat exchange catheter 100 described hereinabove. As described hereinabove with respect to the heat exchange catheter 100, the heat exchange catheter 200 may have a reduced profile relative to conventional heat exchange catheters such that the heat exchange catheter 200 may be placed by non-physician clinicians. For example, an outer diameter of the heat exchange region 250 in its collapsed state may be less than about 12 F, e.g., an outer diameter ranging between about 5 F and about 6 F. The heat exchange catheter 200 may be, e.g., peripherally, placed into the patient, such as in vasculature in the patient's arm or leg.

With reference now to FIGS. 6 and 7, the heat exchange catheter 200 includes a catheter body 210 and the heat exchange region 250 coupled to the catheter body 210. The heat exchange region 250 may be helically wound about the catheter body 210, as shown in FIG. 6, or may have a cylindrical shape disposed about the heat exchange region 250. The catheter body 210 defines a supply lumen 212 and a return lumen 214 separated by an inner wall 260. The inner wall 260 may be formed as an insulating divider to thermally separate the supply lumen 212 and the return lumen 214. The heat exchange region 250 may be formed of a tube or a balloon, as described hereinabove. The catheter body 210 includes the manifold 230 that includes a supply line 232 and a return line 234. The supply line 232 of the manifold 230 is in communication with the supply lumen 212 and connectable to the extracorporeal control system 14 (FIG. 1) via outflow line 32 (FIG. 1) to supply the heat exchange fluid through the supply lumen 212 of the catheter body 210. The return line 234 of the manifold 230 is in communication with the return lumen 214 of the catheter body 210 and connectable to the extracorporeal control system 14 via inflow line 30 (FIG. 1) to return the heat exchange fluid from the heat exchange catheter 210.

The inner wall 260 of the catheter body 210 may be formed of a flexible or elastic material to dynamically enlarge the supply lumen 212 and the return lumen 214. With particular reference to FIG. 7, the inner wall 260 is in a neutral position in the absence of the heat exchange fluid. In the neutral position, the inner wall 260 may be disposed in a generally middle portion of the catheter body 210. In an embodiment, the inner wall 260 in the neutral position may symmetrically (e.g., rotationally or about an axis) divide cross-sections of the supply lumen 212 and the return lumen 214. For example, the inner wall 260 may have a curvature to provide, e.g., symmetric teardrop, cross-sections of the supply lumen 212 and the return lumen 214. However, it is contemplated that the inner wall 260 may be linear in the neutral position.

With reference now to FIG. 8, when the heat exchange fluid is supplied to the supply lumen 212 of the catheter body 210 to initially fill, e.g., the heat exchange region 250 (FIG. 6), the inner wall 260 expands or flexes to enlarge the supply lumen 212, while filling the heat exchange region 250. In order to accommodate the enlarged supply lumen 212 within the catheter body 210, dimensions of the return lumen 214 are reduced.

With reference now to FIG. 9, when the heat exchange fluid is drained from the heat exchange region 250 prior to removal of the heat exchange catheter 200 from the patient, the inner wall 260 is configured to expand or flex to enlarge the return lumen 214 to facilitate drainage of the heat exchange fluid from the heat exchange region 250. In order to accommodate the enlarged return lumen 214 within the catheter body 210, dimensions of the supply lumen 212 are reduced. In this manner, the inner wall 260 may dynamically expand or flex to enlarge the supply or return lumens 212, 214 without enlarging an outer diameter of the catheter body 210. This process is achieved through a pressure differential between the supply lumen 212 and the return lumen 214. In an embodiment, the supply lumen 214 may be clamped and negative pressure may be applied to the return lumen 214 in, e.g., a pulsatile manner (alternately pulling air out and pulling fluid in). In an embodiment, the inner wall 260 in the neutral state may be biased to a configuration shown in FIG. 9, in which the return lumen 214 is enlarged. Such a configuration may facilitate drainage of the heat exchange fluid from the heat exchange region 250. In another embodiment, the return lumen 214 may have a check valve to inhibit reverse flow of the heat exchange fluid into the supply lumen 212.

In an embodiment, the inner wall 260 and the supply and return lumens 212, 214 may be integrally formed as a single construct. In another embodiment, the inner wall 260 and the supply and return lumens 212, 214 may be monolithically formed. The heat exchange region 250 is coupled to the catheter body 210 and in communication with the delivery and return lumens 212, 214 of the catheter body 210 to effect heat exchange with blood of the patient.

The temperature management system 10 cycles the heat exchange fluid as needed to reach the target temperature of the patient. When the desired patient temperature is reached, the temperature management system 10 will slow or stop cycling until the patient's temperature deviates enough from the setpoint or target temperature to require additional warming or cooling. When additional warming or cooling is needed to maintain the setpoint or target temperature, the system will repeat the cycle in/cycle out sequence until the set temperature or target temperature is reached again. Table 1 (shown below) describes exemplary states of various components of the heat exchange system.

TABLE 1

| | | | | | Heat |
|---|---|---|---|---|---|
| | | | | | Exchange |
| | | Console | | Return | Region e.g., |
| State | Description | Actions | Supply Lumen | Lumen | Balloon |
| In Package | Device as delivered | Not connected | Atmospheric | Atmospheric | Fully deflated, |
| | inside sterile barrier | | Pressure | Pressure | folded up and |
| | | | | | sheathed to |
| | | | | | maintain shape for |
| | | | | | ease of insertion |
| Insertion | Placement of the | Not connected to | Atmospheric | Atmospheric | Unsheathed but |
| | catheter into the | catheter, but | Pressure | Pressure | still folded |
| | vasculature through | prepared | | | |
| | venipuncture | | | | |
| Initial | Injection of fluid to | System stops flow | Pressurized with heat | Plugged by console | Inflating and |
| inflation | inflate the balloon. | out the return lumen | exchange fluid. | to allow pressure to | unfolding to full |
| | | and opens flow to | Septum between | build | volume |
| | | supply lumen. | supply and return | | |
| | | Pump pushes heat | lumens of the catheter | | |
| | | exchange fluid into | body flexes into the | | |
| | | supply lumen until | return lumen to | | |
| | | pressure set point | maximize flow of fluid | | |
| | | reached, indicating | into the balloon | | |
| | | balloon has fully | | | |
| | | deployed | | | |
| Indwell | During indwell, | System opens flow | Not | Under vacuum from | Deflates somewhat, |
| cycle out | partial deflation of | to both lumens | pressurized. Septum | console, higher | but not completely |
| | balloon to remove a | Pump pulls vacuum | between lumens rests | pressure in the | |
| | large portion of the | on return lumen | in position to | balloon pushes fluid | |
| | used heat exchange | until a lower limit | maximize cross- | out, back to the | |
| | fluid to send back to | pressure detected on | sectional area of return | console | |
| | the console and | supply side or for a | lumen. Some passive | | |
| | make room for fresh | set time | flow of fresh fluid into | | |
| | fluid | | the balloon is possible | | |
| | | | at a slow rate | | |
| Indwell | During indwell, | System opens flow | Pressurized with heat | Decreased lumen | Inflating |
| cycle in | fresh heat exchange | to both lumens | exchange fluid | cross-sectional area. | |
| | fluid pushed into | Pump pushes heat | Septum between | Allows for passive | |
| | inflow lumen and | exchange fluid into | supply and return | flow back to console | |
| | allowed to flow | inflow lumen until | lumens flexes into the | | |
| | passively out the | pressure set point | return lumen to | | |
| | outflow lumen | reached, or for set | maximize flow of fluid | | |
| | | time | into the balloon | | |
| End of | Deflating the | Shuts off flow to | Closed to flow | Under vacuum. | Deflating |
| treatment | balloon as much as | supply lumen and | Septum between | Maximum cross- | completely to fold |
| | possible to aid in | pulls vacuum on | lumens flexes to | sectional area | as flat as possible |
| | removal at the next | return lumen | minimize supply | | |
| | step | | lumen | | |
| Catheter | Holding vacuum on | When either target | Under vacuum, | Under vacuum, | Fully deflated |
| Withdrawal | the balloon and | pressure is reached | lumen collapsed | lumen maximized | |
| | allowing the | or no flow is | | | |
| | catheter to be | detected for a set | | | |
| | removed from the | time, signals user | | | |
| | patient | safe to withdraw | | | |
| | | Continues to hold | | | |
| | | vacuum with supply | | | |
| | | lumen stopped or | | | |
| | | also under vacuum, | | | |
| | | or blocks flow to | | | |
| | | both lumens | | | |
| Procedure | Catheter has been | User presses button | Atmospheric | Atmospheric | Atmospheric |
| complete | explanted and ready | to indicate catheter | Pressure | Pressure | Pressure |
| | to shut down | has been removed | | | |
| | console | from patient | | | |
| | | Console releases | | | |
| | | inflow and outflow | | | |
| | | Luers | | | |

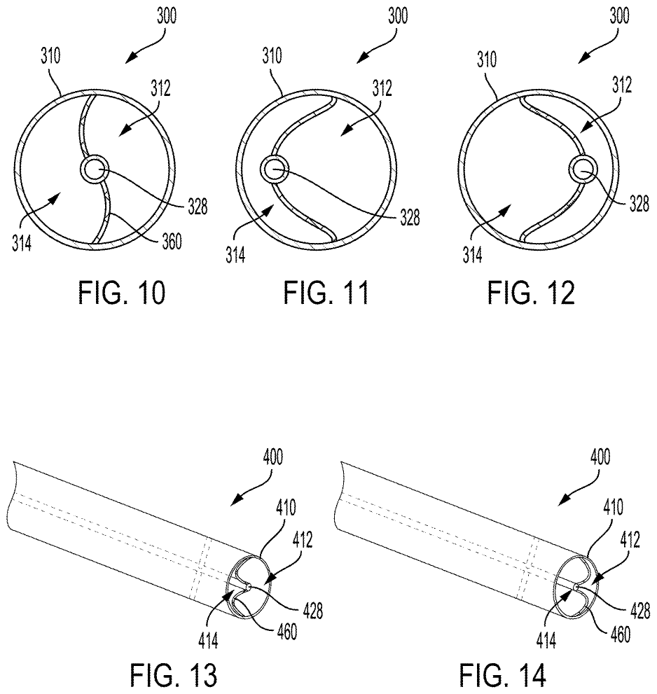

With reference to FIG. 10, another embodiment of a heat exchange catheter is shown as a heat exchange catheter 300. Portions of the heat exchange catheter 300 such as, e.g., a manifold and a heat exchange region, may be similar to the manifold 230 (FIG. 6) and the heat exchange region 250 (FIG. 6) of the heat exchange catheter 200. The heat exchange catheter 300 includes a catheter body 310 and a heat exchange region (not shown) coupled to the catheter body 310. The catheter body 310 includes an inner wall 360 that separates a supply lumen 312 and a return lumen 314. In particular, the catheter body 310 defines a working lumen 328 configured to receive, e.g., a guidewire or medicament, therethrough. In particular, the working lumen 328 and the inner wall 360 may be integrally formed as a single construct such that as shown in FIGS. 11 and 12, the working lumen 328 is movable with the inner wall 360 when the supply lumen 312 or the return lumen 314 are enlarged as shown in FIGS. 11 and 12. In yet another embodiment, a heat exchange catheter 400 includes an inner wall 460 that dynamically expands radially outwards as shown above with respect to the heat exchange catheters 200, 300. However, a working lumen 428 that is configured to receive, e.g., a guidewire or medicament, is stationary relative to an outer wall of the heat exchange catheter 400. In an embodiment, the working lumen 428 may be coupled to an outer wall of the heat exchange catheter 400 at certain locations of the catheter body 410. For example, the working lumen 428 may be coupled to a proximal portion and/or a distal portion of the catheter body 410. Alternatively, the working lumen 428 may be coupled to the outer wall of the catheter body 410 along the length of the catheter body 410. In an embodiment, the working lumen 428 may be concentrically disposed with an outer wall of the catheter body 410. Under such a configuration, the working lumen 428 remains stationary during expansion of the supply lumen 412 when the heat exchange fluid is supplied through the supply lumen 412 to fill the heat exchange region (FIG. 13) and during expansion of the return lumen 414 when the heat exchange fluid is drained from the heat exchange region (FIG. 14) prior to removal of the heat exchange catheter 400 from the patient.

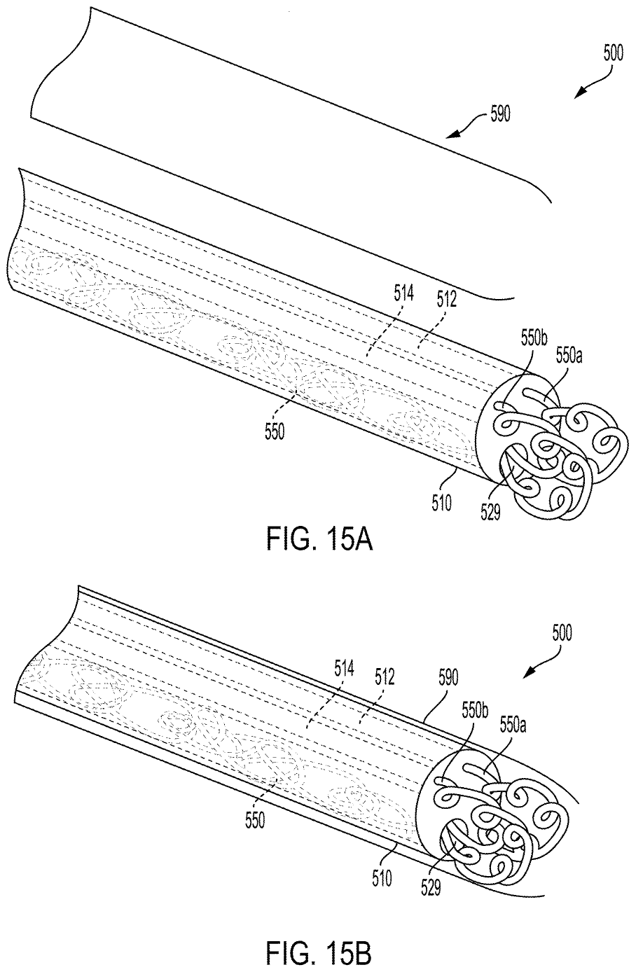

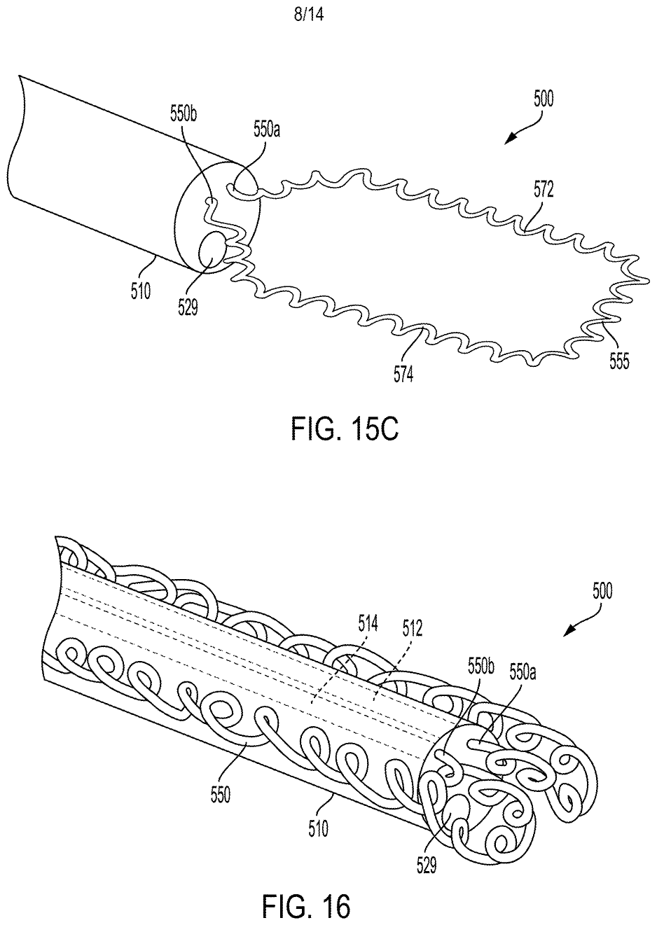

With reference now to FIG. 15A, a heat exchange catheter in accordance with yet another embodiment of the present disclosure is shown generally as a heat exchange catheter 500. The heat exchange catheter 500 may have a reduced profile. Portions of the heat exchange catheter 500 such as, e.g., a catheter body 510 and a manifold, may be similar to the catheter body 110 (FIG. 2) and the manifold 130 (FIG. 2) of the heat exchange catheter 100 described hereinabove. The heat exchange catheter 500 includes a catheter body 510 and a heat exchange region 550 coupled to the catheter body 510. The catheter body 510 includes a supply lumen 512 and a return lumen 514 configured to receive a heat exchange fluid therethrough. The heat exchange region 550 may be an inflatable balloon in communication with the supply and return lumens of the catheter body 510 to receive the heat exchange fluid and thereby effect heat exchange with blood flowing through the vasculature of the patient. The heat exchange region 550 includes a first end 550a that is in communication with the supply lumen 512 of the catheter body 510 and a second end 550b that is in communication with the return lumen 514 of the catheter body 510, whereby the first end 550a is upstream of the second end 550b. The first and second ends 550a, 550b may be coupled to a distal end portion 510b of the catheter body 510.

With continued reference to FIG. 15A, the heat exchange region 550 is transitionable between a deflated state and an inflated state. In the deflated state, the heat exchange region 550 may be positioned proximal to the distal end portion 510b of the catheter body 510. In an inflation state, the heat exchange region 550 may be deployed distally, i.e., distal to the distal end portion 510b of the catheter body 510, such that the heat exchange region 550 extends beyond the distal end portion 510b and floats within the vasculature of the patient while coupled to the distal end portion 510b of the catheter body 510, as shown in FIG. 15C. For example, the heat exchange region 550 does not require a spine, elongate member, catheter body portion or other support structure on which the heat exchange region 550 is supported. In an embodiment, a distal end 555 of the heat exchange region 550 may have a first diameter in the inflated state and the catheter body 510 may have a second diameter larger or smaller than the first diameter. In yet another embodiment, the first and second diameters may be the same.

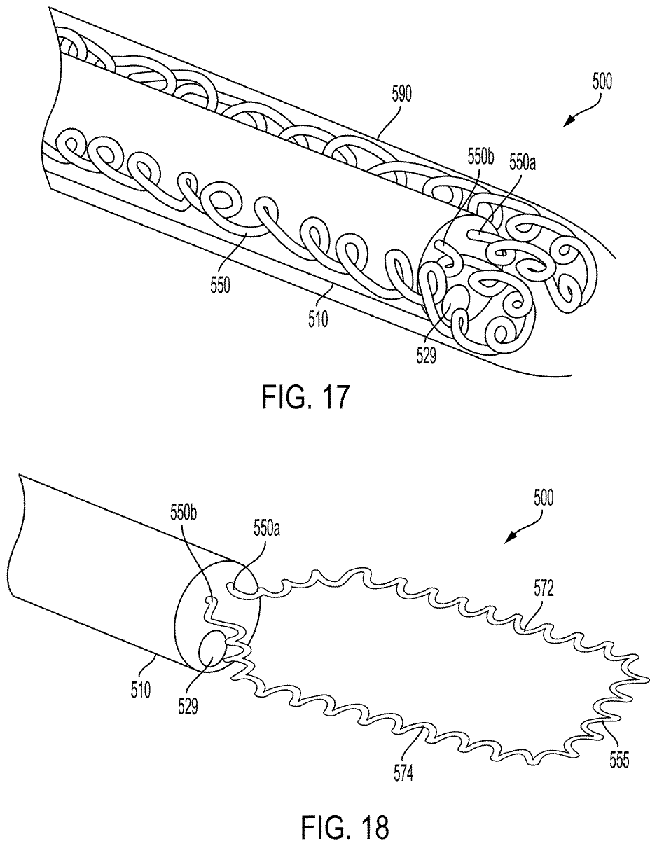

In the deflated state, the heat exchange region 550 may be held within an opening or lumen 529 in the catheter body 510 as shown in phantom in FIG. 15A to reduce the profile of the catheter body 510 and thereby facilitate insertion of the heat exchange catheter 550 into a vasculature of a patient. In an embodiment, the heat exchange region 550 may be deployed through a use of an instrument such as, e.g., a stylet. Alternatively, the heat exchange region 550 may be bonded or coupled to an outer surface 511 of the catheter body 510, as shown in FIG. 16 such that when the heat exchange fluid is supplied to the heat exchange region 550, the heat exchange region 550 is deployed distally while anchored to the catheter body 510 (FIG. 18). In an embodiment, a sleeve, or a sheath 590 (FIGS. 15B and 17) may be provided to cover the heat exchange region 550 in the deflated state in order to facilitate insertion of the heat exchange catheter 500 into the vasculature of the patient and to reduce risk of trauma to the patient. The sleeve 590 may be removed prior to supply of the heat exchange fluid to the heat exchange region 550 such that supply of the heat exchange fluid through the heat exchange region 550 results in deployment of the heat exchange region 550 into the vasculature of the patient.

As the heat exchange region 550 in the deflated state, i.e., placed within the catheter body 510 (FIG. 15A) or bonded or coupled to the outer surface 511 of the catheter body 510 (FIG. 16), is supplied with the heat exchange fluid, the heat exchange region 550 is deployed distally, i.e., distal to the distal end portion 510b of the catheter body 510. As the heat exchange fluid fills the heat exchange region 550 and reaches the threshold pressure, the heat exchange region 550 inflates to a predetermined shape. For example, the heat exchange region 550 may include helical coils that provide an inflow path 572 (running in the distal direction) and an outflow path 574 (returning in the proximal direction), as shown in FIG. 18. However, it is contemplated that the inflated shape of the heat exchange region 550 may be tailored to the required parameters, e.g., heat transfer rate, of the treatment. In this manner, the heat exchange region 550 extends from the distal end portion 510b of the catheter body 510 and floats within the vasculature of the patient. As shown, the heat exchange region 550 may be configured in a series of loops that protrude outwardly from the catheter body 510 so that the blood flowing through the subject's vasculature will pass over and in proximity with the heat exchange region 550, thereby allowing heat to be exchanged between the circulating heat exchange fluid and the subject's flowing blood.

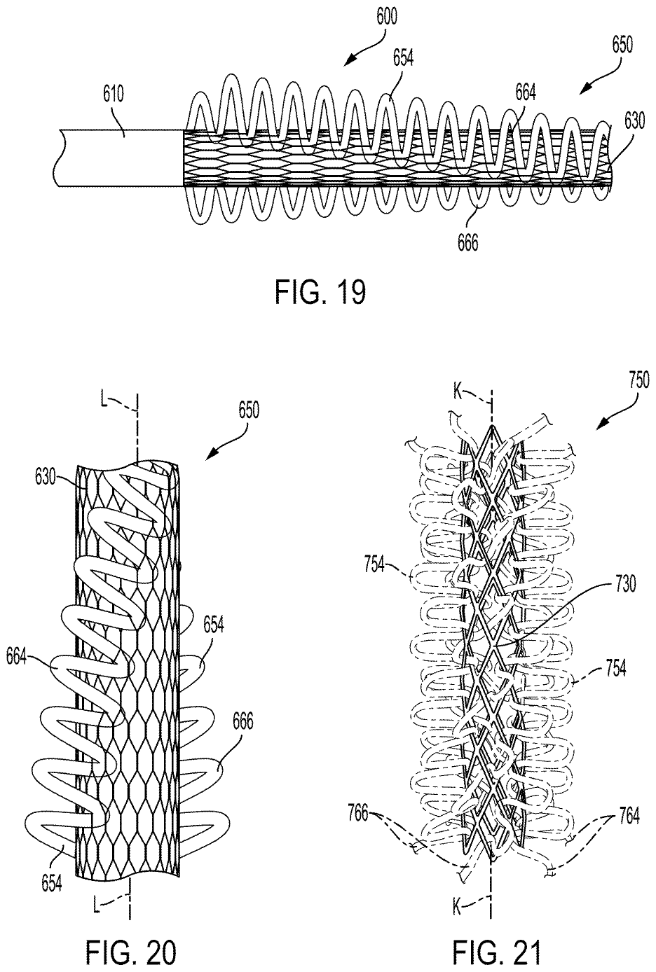

With reference now to FIG. 19, there is provided a heat exchange catheter in accordance with another embodiment of the present disclosure generally shown as a heat exchange catheter 600. The heat exchange catheter 600 includes a heat exchange region 650 configured to reduce thermal shadowing and thereby improve heat exchange between the heat exchange fluid flowing through the heat exchange region 650 and the blood flowing through a vasculature of the patient, as will be described hereinbelow. Portion of the heat exchange catheter 600 such as, e.g., a catheter body 610 and a manifold, may be similar to the catheter body 510 (FIG. 15) of the heat exchange catheter 500 (FIG. 15) and the manifold 130 (FIG. 2) of the heat exchange catheter 100 described hereinabove. The heat exchange catheter 600 includes the catheter body 610 and the heat exchange region 650 configured to receive a heat exchange fluid to exchange heat with blood flowing through the vasculature of the patient. The catheter body 610 includes at least a supply lumen and a return lumen. The heat exchange region 650 is in communication with the supply lumen and the return lumen of the catheter body 610 to receive the heat exchange fluid and thereby effect heat exchange with blood flowing through the vasculature of the patient. The heat exchange region 650 may be, e.g., a tube or a tubular balloon. In the case of an inflatable tubular balloon, supply of the heat exchange fluid causes the inflatable tubular balloon to deploy radially outwards.

The heat exchange region 650 includes a supply fluid path 664 and a return fluid path 666. The supply fluid path 664 receives the heat exchange fluid from the outflow line 32 (FIG. 1) of the extracorporeal control console 14 via the supply lumen of the catheter body 610. The heat exchange fluid returns in the proximal direction through the return flow path 666 of the heat exchange region 650 to the extracorporeal control console 14 via the return lumen of the catheter body 610 and the inflow line 30 (FIG. 1) of the extracorporeal control console 14. However, it is contemplated that both the supply fluid path 664 and the return fluid path 666 may be used to receive the heat exchange fluid from the outflow line 32 of the extracorporeal control console 14 and the heat exchange fluid may return back to the extracorporeal control console 14 via a fluid path provided within the catheter body 610 or vice versa. In an embodiment, a portion of the catheter body 610 may include a mesh 630 disposed about the catheter body 610. Alternatively, a mesh structure may be coupled to the catheter body and extend beyond a distal end of the catheter body.

The heat exchange region 650 may be supported on the mesh 630. In particular, the heat exchange region 650 may be looped through the mesh 630 to create a plurality of heat exchange projections 654 extending radially outwards from the mesh 630. Each heat exchange projection 654 may have a U-shaped profile protruding from the mesh 630. Each heat exchange projection 654 includes a portion that is transverse to a direction of a flow of the blood. The one or more of the heat exchange projections 654 in the same flow path (the supply fluid path 664 or the return fluid path 666) may be angularly offset from each other. However, one or more or all of the heat exchange projections 654 in both the supply and return fluid paths 664, 666 may be angularly offset from each other. In an embodiment, adjacent heat exchange projections 654 or a pair of sequentially arranged heat exchange projections 654 in, e.g., axial sequence, may be angularly offset in a single flow path. A first portion of the plurality of heat exchange projections 654 defines the supply fluid path 664 and a second portion of the plurality of heat exchange projections 654 defines the return fluid path 666. The first portion of the plurality of heat exchange projections 654 and the second portion of the plurality of heat exchange projections 654 may diametrically oppose each other and may define respective helical profiles. The two or more heat exchange projections 654 that define the supply fluid path 664 are axially spaced apart from each other and are angularly offset from each other about an axis “L-L” defined by the catheter body 610. Two or more heat exchange projections 654 that define the supply fluid path 664 may not overlap with each other or may only partially overlap with each other. In an embodiment, adjacent heat exchange projections 654 or a pair of sequentially arranged heat exchange projections 654 define an acute angle. In addition, the adjacent heat exchange projections 654 or the pair of sequentially arranged heat exchange projections 654 are axially displaced along the longitudinal axis “L-L.” Under such a configuration, the heat exchange projections 654 do not impart thermal shadowing on each other or may reduce thermal shadowing of each other and thereby enhance heat exchange between the heat exchange fluid flowing through the heat exchange region 650 and blood flowing through the vasculature of the patient. It is further contemplated that the heat exchange region 650 may be directly supported on the catheter body 610 of the heat exchange catheter 600. For example, the catheter body 610 may define a plurality of bores or openings configured to receive the heat exchange region 650 therethrough such that the heat exchange region 650 is supported on the catheter body 610 without the mesh 630.

Similarly, two or more heat exchange projections 654 that define the return fluid path are axially spaced apart from each other and are angularly offset from each other about the axis “L-L” of the catheter body 610. Two or more heat exchange projections 654 that define the return fluid path 666 may not overlap with each other or may only partially overlap with each other. In an embodiment, adjacent heat exchange projections 654 or a pair of sequentially arranged heat exchange projections 654 of the return fluid path 666 may define an acute angle. In addition, the adjacent heat exchange projections 654 or the pair of sequentially arranged heat exchange projections 654 of the return fluid path 666 may be axially displaced along the longitudinal axis “L-L.” Under such a configuration, the heat exchange projections 654 do not impart thermal shadowing on each other or may reduce thermal shadowing of each other and thereby enhance heat exchange between the heat exchange fluid flowing through the heat exchange region 650 and blood flowing through the vasculature of the patient and coming in contact with the heat exchange region 650.

In an embodiment, the heat exchange fluid flowing through the supply or return fluid path 664, 666 may flow in a general direction of the flow of the blood. Alternatively, the heat exchange fluid flowing through the supply or return fluid path 664, 666 may flow in a general direction opposite of the flow of the blood.

While only a single supply fluid path 664 and a single return fluid path 666 are shown in FIG. 20, it is further contemplated that a plurality of supply fluid paths 764 and/or a plurality of return fluid paths 766 may be provided in a heat exchange region 750 as shown in FIG. 21. (The supply fluid path 764 and return fluid paths 766 are shown as disconnected in FIG. 21 to aid visualization thereof). In particular, the supply fluid paths 764 and the return fluid paths 766 may each have a helical profile. As described hereinabove with respect to the plurality of heat exchange projections 654, the projections 754 of each supply or return fluid path 764, 766 do not overlap each other or may only partially overlap with each other. In particular, two or more projections 754 are axially spaced apart from each other and are angularly offset from each other about an axis “K-K” of the catheter body. In an embodiment, adjacent projections 754 or a pair of sequentially arranged projections 754 defines an acute angle in a single flow path, e.g., supply fluid path 764 or return fluid path 766. In addition, the adjacent projections 754 or the pair of sequentially arranged projections 754 are axially displaced along the direction of the flow of the blood. The projections 754 of the same fluid path do not impart thermal shadowing on each other or may reduce thermal shadowing of each other and thereby enhance heat exchange between the heat exchange fluid flowing through the heat exchange region 750 and blood flowing through the vasculature of the patient. In addition, one or more projections 754 may be angularly offset from one or more projection 754 of a different flow path. The use of the heat exchange catheter 600 may be substantially identical to the use of the heat exchange catheters 100, 100a, 200, 300, 400, 500 described hereinabove, and thus, will not be described herein.

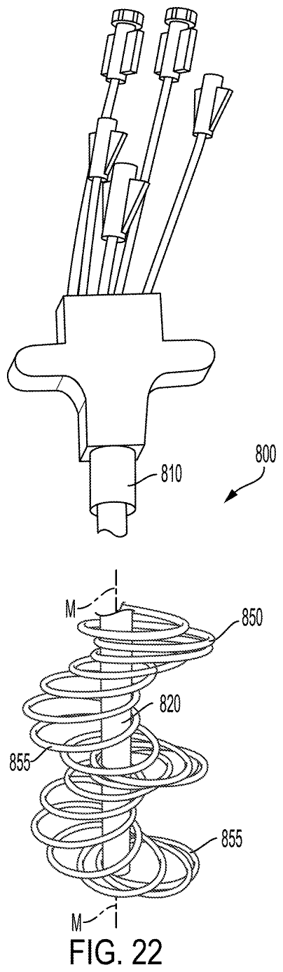

With reference now to FIG. 22, a heat exchange catheter in accordance with another embodiment of the present disclosure is generally shown as a heat exchange catheter 800. The heat exchange catheter 800 includes a catheter body 810, a heat exchange region 850 configured to receive a heat exchange fluid to exchange heat with blood flowing through the vasculature of the patient, and an elongate support 820 coupled to the catheter body 810 and configured to support the heat exchange region 850 thereon. The catheter body 810 includes at least a supply lumen (not shown) and a return lumen (not shown). The heat exchange region 850 is in communication with the supply and the return lumens to enable a heat exchange fluid to flow through the heat exchange region 850. The heat exchange region 850 includes an elongate body in communication with the supply lumen and the return lumen of the catheter body 810 to receive the heat exchange fluid and thereby effect heat exchange with blood flowing through the vasculature of the patient. The elongate body of the heat exchange region 850 may be, e.g., a tube or a tubular balloon. In the case of an inflatable tubular balloon, when a heat exchange fluid is supplied through the inflatable tubular balloon, the inflatable tubular balloon extends radially outwards.

The heat exchange region 850 is in communication with the supply and return lumens of the catheter body 810 to receive a heat exchange fluid and thereby effect heat exchange with blood flowing through the vasculature of the patient. The heat exchange region 850 includes a plurality of first heat exchange portions 855 such that the heat exchange fluid flows through the plurality of first heat exchange portions 855 in a single flow path. One or more first heat exchange portions 855 may have an annular profile and define a plane that is transverse to a direction of a flow of the blood. The plurality of first heat exchange portions 855 defines a supply fluid path. The elongate support 820 may define a conduit (not shown) therein that serves as a return fluid path. Alternatively, the conduit of the elongate support 820 may serve as the supply fluid path and the plurality of first heat exchange portions 855 may define the return fluid path. Alternatively, the plurality of first heat exchange portions 855 may define a supply fluid path and a return fluid path. For example, a first half of the plurality of first heat exchange portions 855 may define the supply fluid path, and a second half of the plurality of first heat exchange portions 855 may define the return fluid path.

In an embodiment, adjacent heat exchange portions 855 or at least a pair of sequentially arranged first heat exchange portions 855, in the same flow path, i.e., the supply fluid path or the return fluid path, or in either path, are axially displaced or angularly offset about an axis “M-M” defined by the elongate support 820. Such a configuration reduces or prevents thermal shadowing, which, in turn, enhances heat exchange between the heat exchange fluid flowing through the heat exchange region 850 and the blood of the patient. In an embodiment, adjacent first heat exchange portions 855 or at least a pair of sequentially arranged first heat exchange portions 855 are angularly offset about the axis “M-M,” but at least partially overlap each other in a superposed relation, which helps reduce thermal shadowing.

In an embodiment, the heat exchange fluid flows through a single flow path in a direction opposite of the direction of the flow of the blood. Alternatively, the heat exchange fluid flows through the single flow path in the direction of the flow of the blood. In an embodiment, the plurality of first heat exchange portions 855 in the same flow path defines a hypotrochoid profile. In another embodiment, the return fluid path may include a plurality of second heat exchange portions that is substantially identical to the plurality of first heat exchange portions 855. The heat exchange fluid may flow through the plurality of second heat exchange portions in a direction opposite of the direction of the flow of the blood. In another aspect, the plurality of second heat exchange portions that are part of the return fluid path is downstream of the plurality of first heat exchange portions 855. The use of the heat exchange catheter 800 is substantially identical to the use of the heat exchange catheters 100, 100a, 200, 300, 400, 500, 600, 700 described hereinabove, and thus, will not be described herein.

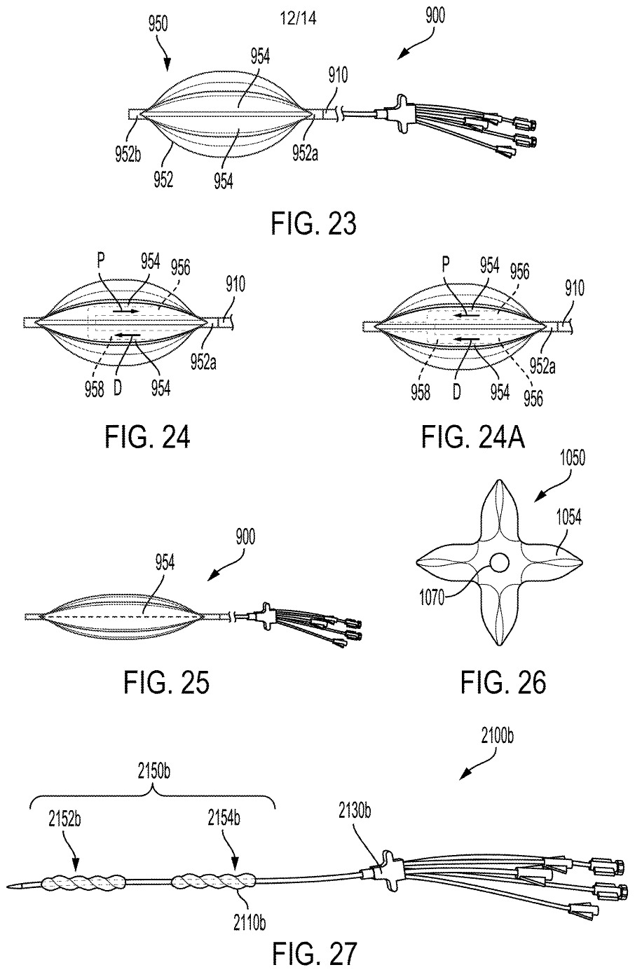

With reference now to FIGS. 23 and 24, a heat exchange catheter in accordance with yet another embodiment of the present disclosure is generally shown as a heat exchange catheter 900. Portions of the heat exchange catheter 900 such as, e.g., a catheter body 910 and a manifold, may be similar to the catheter body 510 (FIG. 15) of the heat exchange catheter 500 and the manifold 130 (FIG. 2) of the heat exchange catheter 100 described hereinabove. The heat exchange catheter 900 includes a catheter body 910 and a heat exchange region 950 configured to receive a heat exchange fluid to exchange heat with blood flowing through the vasculature of the patient. The catheter body 910 includes at least a supply lumen (not shown) and a return lumen (not shown). The heat exchange region 950 is in communication with the supply and the return lumens to enable a heat exchange fluid to flow through the heat exchange region 950. The heat exchange region 950 includes a body 952 having e.g., an oval shape. The body 952 includes a plurality of ridges 954 disposed about the body 952.

With particular reference to FIG. 24, in an embodiment, the plurality of ridges 954 may be in fluid communication. Each ridge 954 extends between proximal and distal end portions 952a, 952b of the body 952. In an embodiment, each ridge 954 includes a conduit 956 in communication with the supply and return lumens of the catheter body 910 to receive the heat exchange fluid therethrough. A connecting passageway 958 may be provided between adjacent or neighboring ridges 954 to connect the conduits 956 of adjacent ridges 954. In an embodiment, the heat exchange fluid flows through neighboring ridges 954 in opposite directions. In another embodiment, the heat exchange fluid may travel through the ridges 954 in parallel and converged into a return conduit that is connected to the return lumen of the catheter body 910, as shown in FIG. 24A. In addition, the rounded contours of the ridges 954 allow the heat exchange region 950 to maintain a relatively atraumatic profile, thereby minimizing the possibility of damage to the blood vessel wall. A heat exchange region 950 may include two, three, or more ridges 954. The heat exchange region 950 may be an inflatable balloon that is transitionable between a deflated state having a reduced profile (FIG. 25) and an inflated state (FIG. 23) in which the ridges 954 of the heat exchange region 950 expand radially outwards. Under such a configuration, when the heat exchange region 950 is positioned in the vasculature of the patient, the heat exchange fluid circulating through the heat exchange region 950 exchanges heat with the blood flowing through the vasculature of the patient. The heat exchange region 950 may provide increased heat exchange surface compared to, e.g., a heat exchange region having a cylindrical structure, which, in turn, may increase heat exchange between the heat exchange region 950 and the blood.

With reference to FIG. 26, in an embodiment, a heat exchange region 1050 includes ridges 1054 that are similar to the ridges 954 described hereinabove. However, the number of ridges 1054 may be tailored to meet the requirements of the treatment. In an embodiment, the heat exchange region 1050 may define a working lumen 1070 configured to receive, e.g., a guidewire or medicament, therethrough. The use of the heat exchange catheter 900 is substantially identical to the above-described heat exchange catheters.

With reference to FIG. 27, there is illustrated a heat exchange catheter 2100b for use with the temperature management system 10 (FIG. 1) in accordance with yet another embodiment of the present disclosure. Portions of the heat exchange catheter 2100b such as, e.g., a catheter body 2110b and a manifold 2130b, may be similar to the catheter body 110 (FIG. 2) and the manifold 130 (FIG. 2) of the heat exchange catheter 100 described hereinabove. The heat exchange catheter 2100b includes a catheter body 2110b and a heat exchange region 2150b supported about the catheter body 2110b. The heat exchange region 2150b may include one or more segments. For example, the heat exchange region 2150b may be divided into a first segment 2152b and a second segment 2154b. Each segment 2152b, 2154b includes a tubing or a tubular balloon that is helically wound about the catheter body 2110b. The first and second segments 2152b, 2154b may be axially displaced. In an embodiment, the heat exchange region 2150b may have a length of about 4-6 inches. In an embodiment, the first segment 2152b and the second segment 2154b may each have a length of about 2-3 inches. In an embodiment, the catheter body 2110b may have a length of about 7-19 inches. In another embodiment, the catheter body 2110b may have a length of about 13-15 inches. In an embodiment, the first and second segments 2152b, 2154b may be formed as a single continuous segment rather than two spaced apart segments In another embodiment, the first and second segments 2152b, 2154b may molded onto the catheter body 2110b, which facilitates manufacturability of the heat exchange catheter 2100b. Alternatively, the tubing or the tubular balloon may extend through a plurality of openings as shown in FIG. 4 and/or be wrapped around the catheter body or supported on the catheter body 2110b in a helical profile as shown in FIG. 5A. The heat exchange catheter 2100b may or may not provide a bypass configuration to bifurcate the heat exchange fluid supplied to the supply lumen of the catheter body 2110b.

The heat exchange power output of the heat exchange catheter 2110b (FIG. 27) was compared with the heat exchange power output of a heat exchange catheter including a heat exchange region having two cylindrical heat exchange regions supported about a catheter body of the heat exchange catheter. Such heat exchange catheter had an outer diameter (OD) of about 9.3 F and a heat exchanger region length of about 22 cm. Test results revealed that the heat exchange power output of the heat exchange catheter 2110b was about 20% greater than the heat exchange power output of the heat exchange catheter having the two cylindrical shaped heat exchange regions, as shown in Table 2 below. In addition, the design of the heat exchange region 2150b of the heat exchange catheter 2100b also improves manufacturability while improving heat exchange power output. Further, such a design may reduce blood thrombogenicity.

TABLE 2

| | | | Catheter | Catheter | | |
|---|---|---|---|---|---|---|
| | Peak | Circulation | Input | Output | Flow | Power |
| | Pressure | Temperature | Temperature | Temperature | Rate | Output |
| | (PSI) | (° C) | (° C) | (° C) | (mL/min) | (Watts) |
| Heat Exchange | 47 | 37 | 4.14 | 10.73 | 210 | 98 |
| Catheter 2100b | | | | | | |
| Catheter w/ | 52.5 | 37 | 4.19 | 9.34 | 210 | 77 |
| Two | | | | | | |
| Cylindrical | | | | | | |
| Heat Exchange | | | | | | |
| Regions | | | | | | |

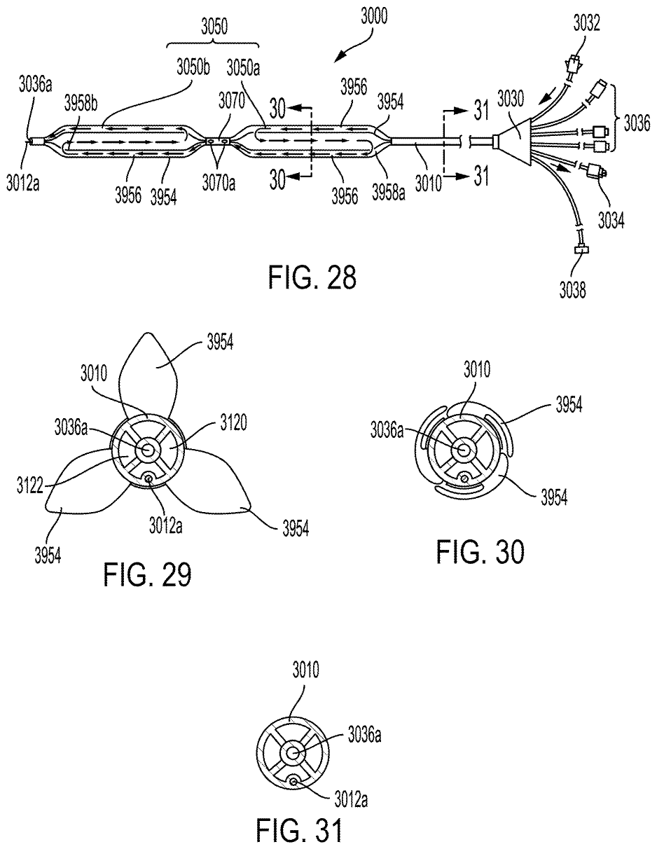

With reference now to FIGS. 28 and 29, a heat exchange catheter in accordance with yet another embodiment of the present disclosure is generally shown as a heat exchange catheter 3000. Portions of the heat exchange catheter 3000 such as, e.g., a catheter body 3010 and a manifold 3030, may be similar to the catheter body 910 (FIG. 23) of the heat exchange catheter 900 and the manifold 130 (FIG. 2) of the heat exchange catheter 100 described hereinabove. In an embodiment, a portion of the manifold 3030 may be overmolded onto the catheter body 3010. The manifold 3030 may include an inflow connector 3032, an outflow connector 3034, one or more infusion lines 3036, and a temperature probe connector 3038. The inflow connector 3032 of the manifold 3030 is in communication with the delivery lumen 3120 of the catheter body 3010 and is connectable to the extracorporeal control console 14 via an outflow line 32 (FIG. 1) to supply the heat exchange fluid through the heat exchange region 3050 of the heat exchange catheter 3000. The outflow connector 3034 of the manifold 3030 is in communication with the return lumen 3122 of the catheter body 3010 and is connectable to the extracorporeal control console 14 via an inflow line 30 (FIG. 1) to return the heat exchange fluid to the extracorporeal control console 14 from the heat exchange region 3050 of the heat exchange catheter 3000. The one or more infusion lines 3036 are in communication with one or more working lumens or working channels of the catheter body 3010.

At least one working lumen may serve as a guidewire lumen 3036a to receive a guide wire to facilitate insertion and positioning of the heat exchange catheter 3000 and/or may be used for delivery of fluids, medicaments, or other devices. The heat exchange catheter 3000 is a low profile catheter, e.g., up to about 9-10 Fr, which may be inserted through, e.g., a central venous/arterial system or vasculature. The low profile heat exchange catheter 3000 includes a heat exchange region 3050 having a plurality of ridges 3954 that provides a greater heat exchange surface area than, e.g., a heat exchange catheter having a cylindrical heat exchange region with no ridges. The heat exchange catheter 3000 may be formed of copolymers such as, e.g., Pebax® elastomers or other medical grade materials such as, e.g., polyurethane and nylon. In an embodiment, a heat exchange region 3050 may be formed of Pebax® elastomers or Chronoprene® thermoplastic elastomer. It is also envisioned that the heat exchange region may have a single ridge that extends non-concentrically from the catheter body 3010.

In an embodiment, the heat exchange catheter 3000 may include a temperature and/or location sensing capability. For example, a temperature and/or location sensor may be inserted through the guidewire lumen 3036a and disposed at any position along the catheter body 3010. Alternatively, the temperature and/or location sensor may be advanced out of a distal end opening of the guidewire lumen 3036a to a location beyond a distal end of the catheter body 3010. In an embodiment, the temperature and/or location sensor may have, e.g., a soft and/or tapered tip, which aids insertion. The temperature sensor enables a clinician to measure the patient's blood temperature while using the heat exchange catheter 3000 which is connected to the extracorporeal control console 14 for tracking/displaying patient temperature. Additionally, the location sensor capability of a separate or combined location sensor enables the clinician to navigate the catheter tip while avoiding certain anatomical structures such as, e.g., an atrium or ventricle of the heart. In an embodiment, the tip may include a contact force sensing mechanism or magnetic transmitter/locator. A temperature sensor and/or location sensor may be integral with or advanceable through the catheter.

The heat exchange catheter 3000 includes the catheter body 3010 and a heat exchange region 3050 configured to receive a heat exchange fluid to exchange heat with blood flowing through the vasculature of the patient. The catheter body 3010 includes the delivery lumen 3120 and the return lumen 3122. The heat exchange region 3050 includes first and second segments 3050a, 3050b. The first and second segments 3050a, 3050b are coupled by a connecting portion 3070 defining infusion holes 3070a in communication with the delivery lumen 3120 to supply, e.g., medicament, to the patient. While two segments of the exchange region are shown in FIG. 28, in certain embodiments, a heat exchange region may include one or more segments. If a single heat exchange region segment is provided, there would be no connecting portion, and infusion holes may be located distal or proximal to the heat exchange region.

The heat exchange region 3050 is in communication with the delivery and return lumens 3120, 3122 to enable a heat exchange fluid to flow through the heat exchange region 3050. Each of the first and second segments 3050a, 3050b includes one or more ridges 3954 that are disposed about the catheter body 3010. In an embodiment, there may be three ridges 3954 that are uniformly arranged about the catheter body 3010. In an embodiment, the plurality of ridges 3954 may be a balloon formed of, e.g., an elastomer. In another embodiment, the catheter body 3010 may also be formed of, e.g., an elastomer. The plurality of ridges 3954 may be, e.g., laser or heat, bonded to the catheter body 3010.

Each ridge 3954 extends longitudinally between proximal and distal portions of the corresponding first or second segment 3050a, 3050b. Further, each ridge 3954 is in fluid communication with one or more other ridges 3954 in the corresponding first or second segment 3050a, 3050b. In an embodiment, each ridge 3954 also serves as a conduit 3956 (FIG. 28) in communication with the delivery and return lumens 3120, 3122 of the catheter body 3010 to receive the heat exchange fluid therethrough. A connecting passageway 3958a, 3958b may be provided between adjacent or neighboring ridges 3954 to provide fluid connection therebetween. The heat exchange fluid may flow through adjacent ridges 3954 in series, e.g., in alternating opposite directions. Under such a configuration, the heat exchange fluid is supplied to a ridge 3954 of the first segment 3050a through the delivery lumen 3120. The heat exchange fluid then flows sequentially through all of the other ridges 3954 in the first segment 3050a and is directed to a delivery lumen of the connecting portion 3070. Thereafter, the heat exchange fluid enters the second segment 3050b and flows sequentially through all the ridges 3954 of the second segment 3050b. The return lumen 3122 is in communication with at least one of the ridges 3954 of the second segment 3050b such that the heat exchange fluid flows back to the extracorporeal control console 14 from the second segment 3050b.

Alternatively, the heat exchange fluid may travel through the ridges 3954 of the first segment 3050a in parallel and at least a portion of the heat exchange fluid may converge into a return conduit that is connected to the return lumen 3122 of the catheter body 3010 such that the portion of the heat exchange fluid flows back to the extracorporeal control console from the first segment 3050a. The remaining portion of the heat exchange fluid may be directed to the ridges 3954 of the second segment 3050b through the connecting portion 3070. The remaining portion of the heat exchange fluid may flow through the ridges 3954 of the second segment 3050b in parallel or in series, as described hereinabove.

The heat exchange region 3050 with the ridges 3954 may include one or more inflatable balloons that are transitionable between a deflated state having a reduced profile (FIG. 30) and an inflated state (FIG. 29) in which the ridges 3954 of the heat exchange region 3050 expand radially outwards. Prior to inflation, the ridges 3954 of the heat exchange region 3050 may be folded or wrapped, e.g., around the catheter body 3010, such that portions of the ridges 3954 overlap to create a low profile and a smooth surface to facilitate insertion. Under such a configuration, insertion may be performed without requiring an introducer sheath.

When the heat exchange region 3050 is positioned in the vasculature of the patient, the heat exchange fluid circulating through the heat exchange region 3050 exchanges heat with the blood flowing through the vasculature of the patient. The heat exchange region 3050 may provide increased heat exchange surface compared to, e.g., a heat exchange region having a cylindrical structure, with no ridges, which, in turn, may increase heat exchange between the heat exchange region 3050 and the blood.

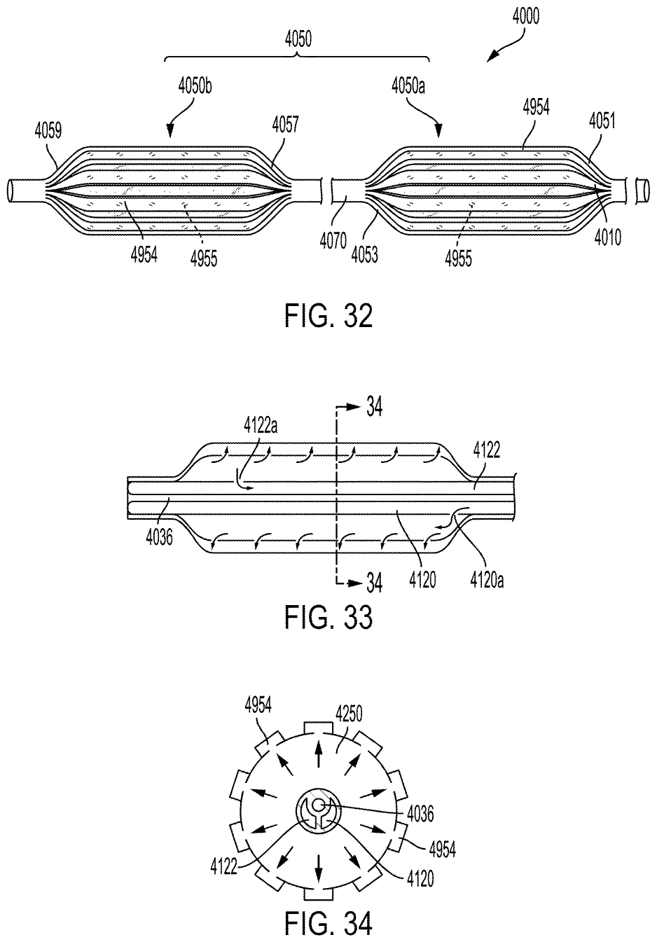

With reference now to FIGS. 32-34, a heat exchange catheter in accordance with yet another embodiment of the present disclosure is generally shown as a heat exchange catheter 4000. Portions of the heat exchange catheter 4000 such as, e.g., a catheter body 4010 and a manifold, may be similar to the catheter body 3010 (FIG. 28) and the manifold 3030 (FIG. 28) of the heat exchange catheter 3000 described hereinabove. The heat exchange catheter 4000 includes the catheter body 4010 and a heat exchange region 4050 configured to receive a heat exchange fluid to exchange heat with blood flowing through the vasculature of the patient. The catheter body 4010 includes a delivery lumen 4120, a return lumen 4122, and a working lumen 4036. The heat exchange region 4050 includes first and second segments 4050a, 4050b. The first and second segments 4050a, 4050b are coupled by a connecting portion 4070. In an embodiment, the connecting portion 470 may define infusion holes in communication with the working lumen 4036 to supply, e.g., medicament, to the patient. While two segments of the exchange region are shown in FIG. 32, in certain embodiments, a heat exchange region may include one or more segments. If a single heat exchange region segment is provided, there would be no connecting portion and infusion holes may be located distal or proximal to the heat exchange region.

The delivery lumen 4120 is connectable to the extracorporeal control console 14 via an outflow line 32 (FIG. 1) to supply the heat exchange fluid through a heat exchange region 4050 of the heat exchange catheter 4000. An outflow connector of the manifold is connectable to the return lumen 4122 of the catheter body 4010 and the extracorporeal control console 14 via an inflow line 30 (FIG. 1) to return the heat exchange fluid to the extracorporeal control console 14.

The heat exchange region 4050 is in communication with the delivery and return lumens 4120, 4122 to enable a heat exchange fluid to flow through the heat exchange region 4050. Each of the first and second segments 4050a, 4050b includes an inflatable chamber 4250 or cavity (FIG. 34) and one or more ridges 4954 that are arranged about the inflatable chamber 4250 or extend from the surface of the chamber. In an embodiment, a plurality of ridges 4954 may be uniformly arranged about the inflatable chamber 4250. The ridges 4954 are in fluid communication with the inflatable chamber 4250 such that when the heat exchange fluid is supplied to the inflatable chamber 4250, the heat exchange fluid is also supplied to the ridges 4954 and extends the ridges 4954 radially outwards. In an embodiment, each ridge 4954 may define one or more of openings 4955 to provide fluid communication between the ridge 4954 and the inflatable chamber 4250. In an embodiment, the inflatable chamber 4250 and/or the ridges 4954 may be an expandable balloon formed of, e.g., an elastomer. In another embodiment, the body 4010 may also be formed of, e.g., an elastomer or polyurethane. The ridges 4954 and the inflatable chamber 4250 may be formed as a single construct. In an embodiment, the ridges 4954 and the inflatable chamber 4250 may be monolithically formed.

Each ridge 4954 extends longitudinally between proximal and distal portions of the corresponding first or second segment 4050a, 4050b. The catheter body 4010 may define a first opening 4120a adjacent proximal portions 4051 of the respective first and second segment 4050a, 4050b such that the first opening 4120a is configured to provide fluid communication between the delivery lumen 4120 and the inflatable chamber 4250 to supply the heat exchange fluid to the inflatable chamber 4250. In an embodiment, the body 4010 may further define a second opening 4122a such that the second opening 4122a is configured to provide fluid communication between the inflatable chamber 4250 and the return lumen 4122 to return the heat exchange fluid to the extracorporeal control console 14. Under such a configuration, a portion of the heat exchange fluid flowing through the delivery lumen 4120 enters the first segment 4050a of the heat exchange region 4050 and returns back to the extracorporeal control console 14, and the remaining heat exchange fluid flows through the second segment 4050b of the heat exchange region 4050 before returning back to the extracorporeal control console 14. Under such a configuration, the heat exchange fluid may flow through the ridges 4954 of the first segment 4050a in series or in parallel, as described hereinabove. Similarly, the heat exchange fluid may flow through the ridges 4954 of the second segment 4050b in series or in parallel, as described hereinabove. It is further contemplated that the heat exchange fluid may flow through the ridges 4954 of the first segment 4050a in series and flow through the ridges 4954 in the second segment 4050b in parallel or vice versa. Alternatively, an opening may be placed adjacent the distal portion 4053 of the first segment 4050a. The opening may provide fluid communication between the inflatable chamber 4250 and the delivery lumen 4120 such that the heat exchange fluid flowing through the first segment 4050a of the heat exchange region 4050 is directed into the second segment 4050b without any of the heat exchange fluid returning to the extracorporeal control console 14 until after the fluid flows through the second segment 4050b. Under such a configuration, the heat exchange fluid may flow through the ridges 4954 of the first segment 4050a in series or in parallel. Similarly, the heat exchange fluid may flow through the ridges 4954 of the second segment 4050b in series or in parallel. It is further contemplated that the heat exchange fluid may flow through the ridges 4954 of the first segment 4050a in series and through the ridges 4954 of the second segment 4050b in parallel or vice versa.

Similarly, a portion of the body 4010 corresponding to the second segment 4050b of the heat exchange region 4050 may define an opening or hole 4120a adjacent a proximal portion 4051 of the second segment 4050b. The opening 4120a is configured to provide fluid communication between the delivery lumen 4120 and the inflatable chamber 4250 of the second segment 4050b to supply the heat exchange fluid to the inflatable chamber 4250. Further, the body 4010 may further define an opening or hole 4122a adjacent a distal portion of the second segment 4050b. The opening 4122a is configured to provide fluid communication between the inflatable chamber 4250 and the return lumen 4122. The openings 4120a and 4122a may be disposed at opposite ends of the second segment 4050b.

Under such a configuration, when the heat exchange region 4050 is positioned in the vasculature of the patient, the heat exchange fluid circulating through the heat exchange region 4050 exchanges heat with the blood flowing through the vasculature of the patient. The heat exchange region 4050 may provide increased heat exchange surface compared to, e.g., a heat exchange region having a cylindrical structure with no ridges, which, in turn, may increase heat exchange between the heat exchange region 3050 and the blood.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of embodiments have been described. For example, the detailed description and the accompanying drawings to which it refers are intended to describe some, but not necessarily all, examples or embodiments of the system. The described embodiments are to be considered in all respects only as illustrative and not restrictive. Nevertheless, it will be understood that various modifications may be made without departing from the spirit and scope of the data processing system described herein. Accordingly, other embodiments are within the scope of the following claims.

1. A heat exchange catheter insertable into a vasculature of a patient, the heat exchange catheter comprising:
a catheter body, the catheter body comprising:
a delivery lumen;
a return lumen, the delivery lumen and the return lumen configured to receive a working fluid therethrough;
an inner wall separating the delivery and return lumens, the inner wall defining an opening in communication with the delivery and return lumens; and
a side wall defining at least one opening in communication with the delivery lumen; and
a heat exchange region, the heat exchange region comprising a first end and a second end, the first end of the heat exchange region coupled to the delivery lumen of the catheter body through the at least one opening,
wherein, the heat exchange region is configured to be in fluid communication with the delivery and return lumens of the catheter body to receive the working fluid and thereby effect heat exchange with blood flowing through the vasculature of the patient, and
wherein the opening of the inner wall is located such that the working fluid flowing through the delivery lumen of the catheter body is bifurcated between the heat exchange region and the return lumen.

2. The heat exchange catheter according to claim 1, wherein the opening in the inner wall is upstream of the heat exchange region.

3. The heat exchange catheter according to claim 2, wherein the heat exchange region projects through the at least one opening such that the heat exchange region is supported about the catheter body.

4. The heat exchange catheter according to claim 2, wherein the working fluid flowing through the heat exchange region bypasses a portion of the delivery lumen of the catheter body.

5. The heat exchange catheter according to claim 4, wherein the catheter body further includes a pair of plugs disposed in the delivery lumen to establish a fluid-tight seal against the delivery lumen such that the working fluid is directed through the heat exchange region.

6. The heat exchange catheter according to claim 5, wherein the catheter body further defines a working lumen dimensioned to receive a guidewire therethrough.

7. The heat exchange catheter according to claim 1, wherein the catheter body has a diameter ranging between about 5 Fr and about 6 Fr.

8. The heat exchange catheter according to claim 1, wherein the heat exchange region is an inflatable balloon.

9-49. (canceled)

50. A heat exchange catheter insertable into a vasculature of a patient, the heat exchange catheter comprising:
a catheter body including a delivery lumen, a return lumen, and an inner wall separating the delivery and return lumens configured to receive a working fluid therethrough, the inner wall defining at least one opening;
a heat exchange region configured to be in fluid communication with the delivery and return lumens of the catheter body to receive the working fluid and thereby effect heat exchange with blood flowing through the vasculature of the patient, and
wherein the at least one opening of the inner wall is defined proximal of the heat exchange region such that a portion of the working fluid supplied through the delivery lumen of the catheter body flows proximally through the return lumen prior to reaching the heat exchange region.

51. The heat exchange catheter according to claim 50, wherein the catheter body further includes a side wall defining at least one opening in communication with the delivery lumen.

52. The heat exchange catheter according to claim 51, wherein the heat exchange region projects through the at least one opening of the side wall such that the heat exchange region is supported about the catheter body.

53. The heat exchange catheter according to claim 52, wherein the working fluid flowing through the heat exchange region bypasses a portion of the delivery lumen of the catheter body.

54. The heat exchange catheter according to claim 53, wherein the catheter body further includes a pair of plugs disposed in the delivery lumen to establish a fluid-tight seal against the delivery lumen such that the working fluid is directed through the heat exchange region.

55. The heat exchange catheter according to claim 50, wherein the catheter body further defines a working lumen dimensioned to receive a guidewire therethrough.

56. The heat exchange catheter according to claim 55, wherein the catheter body has a diameter ranging between about 5 Fr and about 6 Fr.

57. The heat exchange catheter according to claim 50, wherein the heat exchange region is a tubular balloon.
